(19) United States
(12) Patent Application Publication
(10) Pub. No.: US 2021/0200753 A1
(43) Pub. Date: Jul. 1, 2021

(54) Strain Recommendation System and Method

(71) Applicant: StrainTech Inc., Los Angeles, CA (US)

(72) Inventors: Alec Perelman, Los Angeles, CA (US); Serhii Parkhomenko, Warsaw (PL); Micky Dionisio, Moorpark, CA (US)

(73) Assignee: StrainTech Inc., Los Angeles, CA (US)

(21) Appl. No.: 17/198,053

(22) Filed: Mar. 10, 2021

Related U.S. Application Data
(63) Continuation-in-part of application No. 15/369,842, filed on Dec. 5, 2016, now abandoned.

(51) Int. Cl. G06F 16/248; G16H 20/10; A01H 1/04

(52) U.S. Cl. CPC G06F 16/248; A01H 1/04; G16H 20/10

(57) ABSTRACT

Machine(s), method(s), and media involve receiving at least one network-based communication regarding strain-related characterizing selections; generating at least one ad hoc matching calculation weighting system based at least in part upon the receiving at least one network-based communication regarding strain-related characterizing selections; using the at least one ad hoc matching calculation weighting system with strain candidate data stored in the at least one database to at least in part determine at least one strain candidate score for at least one strain candidate; and outputting at least one network-based communication regarding at least one strain recommendation based at least in part on the at least one strain candidate score. In addition, other aspects are described in the claims, drawings, and text forming a part of the present disclosure.

### BACKGROUND

Over millennia, various herbs (e.g., plants, herbs, and/or crops including biologically produced substances) have been cultivated and selectively bred to increase a desired effect upon humans and living creatures (living beings) caused by application of the cultivated plant. The various herbs are typically selectively bred to increase (and/or introduce) desired concentrations of active ingredients that are produced by and contained in a particular cultivated plants so that, for example, the cultivated plant's effectiveness as a medicine, restorative agent, mood enhancer, and the like on a living being is increased. Various systems have been proposed for classifying such cultivated herbs (including, for example, genus-species-variety-cultivar taxonomies). However, the fecundity of genetic variation of cultivated herbs and differences in outcomes in various modes of the application of the cultivated herbs increases the difficulty of identifying a source for procuring a cultivated herb for a particular desired outcome.

### BRIEF DESCRIPTION OF THE DRAWINGS

FIG. 1 is a network diagram showing a network suitable for practicing aspects of selection and/or finding of desired cultivated strains in accordance with exemplary embodiments of the disclosure.

FIG. 2 shows an illustrative computing device 200 in accordance with exemplary embodiments of the disclosure.

FIG. 3 is a network diagram showing a network for selection and/or finding of desired cultivated strains in accordance with exemplary embodiments of the disclosure.

FIG. 4 is a logic diagram showing a system for selection and/or finding of desired cultivated strains in accordance with exemplary embodiments of the disclosure.

FIG. 5 is a logic diagram of a strain knowledge base 500 in accordance with embodiments of the present disclosure.

FIG. 6 is a logic diagram of a customer information manager 600 in accordance with embodiments of the present disclosure.

FIG. 7 is a logic diagram of a vendor information manager 700 in accordance with embodiments of the present disclosure.

FIG. 8 is a logic diagram of a strain vendor locator 800 in accordance with embodiments of the present disclosure.

FIG. 9 is a signal flow diagram of a strain product finder in accordance with embodiments of the present disclosure.

FIG. 10 is a flow diagram of a process for finding strain products in accordance with embodiments of the present disclosure.

### DETAILED DESCRIPTION

The following discussion is directed to various exemplary embodiments of the disclosure. Although one or more of these exemplary embodiments may be preferred, the exemplary embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description—and claims—to refer to particular system components. As one skilled in the art will appreciate, various names may be used to refer to a component. Accordingly, distinctions are not necessarily made herein between components that differ in name but not function. In the following discussion and in the claims, the terms “including” and “comprising” are used in an open-ended fashion, and thus are to be interpreted to mean “including, but not limited to . . . .” Also, the terms “coupled to” or “couples with” (and the like) are intended to describe either an indirect or direct electrical, optical and/or wireless connection. Thus, if a first device couples to a second device, that connection can be made through a direct connection, or through an indirect connection via other devices and connections.

The term “human” can, in accordance with principles of broad applicability (e.g., metonymy), include the meaning of non-human animals unless the context clearly indicates otherwise. The term “illness” can include the meaning of a biological state having symptoms associated with a particular illness. The term “symptom” can include the meaning of a result of an illness and/or an outcome (e.g., physiological and/or psychological effect) of the application of a particular strain. The term “strain” can refer to a cultivar (or other named term from another nomenclature where variations in outcomes are determined for a plant identified by the named term), genetically identical crops grown under varying conditions (accordingly having different concentrations of active ingredients), and/or genetically diverse crops having a substantially similar range of outcomes (e.g., substantially similar concentrations of active ingredients). The term “strain” can mean (e.g., in accordance with principles of metonymy) one or more “strain products.” The term “strain product” can refer to a cultivar, a strain, a formulation of one or more parts of one or more strains and/or cultivars, a concentrate of a cultivar and/or strain, a portion of a cultivar, selected portions of plants of the same or heterogeneous cultivars, formulations having active ingredients (e.g., from one or more of classes of drugs including any of the below-described THC, CBD, CBN, and the like) of strains (regardless of whether the active ingredients are derived from naturally grown plants or synthetically produced), and the like.

The disclosed strain product finder includes apparatuses and methods arranged, for example, to receive a strain product search request from a particular customer. A list of highest matching strain products is generated in response to searching a strain objects database for strain products to determine highest matching strain products by comparing each of the sets of stored ranked outcomes with a set of ranked outcomes determined for the customer. At least on vendor dispensary for dispensing at least one of the highest matching strain products is selected in response to querying a vendor inventory. Search results are sent to the customer, wherein the search results are determined in response to a location of each of the selected vendor dispensary and a customer location associated with the particular customer

One area in which herbs are being selectively bred and cultivated includes applications of cannabis, and especially medical cannabis (e.g., medical marijuana). Presently, thousands of strains of cannabis are being bred and classified at increasingly faster rates. Genetic diversity and differences in production methods often result in substantially differing concentrations of active ingredients even within plants commonly classified as a particular named cultivar and/or strain. Further, (e.g., one or more portions of) one or more (e.g., same or different) cultivars are selectively combined to produce various strain products such as concentrates, edibles, teas, buds, oils, lotions, formulations of particular concentrations of active ingredients for a specific medical prescription, formulations of particular concentrations of active ingredients in accordance with (e.g., locally applicable) laws, rules, regulations, and the like (e.g., legality of a jurisdiction).

Accordingly, patients (e.g., living beings for whom application of a cannabis strain is intended) are encountering increasing difficulties in determining accurate information about a strain (or strain product) as well as information about the strain's effects. Further, individual patients can have a wide variety of intended uses for a strain product and seek differing expected outcomes, which further increases the difficulty of selecting a particular strain for a particular purpose (e.g., selected and/or prescribed outcome).

As disclosed herein, various outcomes are associated with a use of a particular strain product, which facilitates a patient (including patient surrogate and/or care provider) more easily and accurately identifying a desired strain and locating (e.g., determine a locally situate provider/dispensary) a source for obtaining the desired strain. Patients optionally can directly provide information concerning personally experienced outcomes with a desired strain so that, for example, similar strains to the desired strain can be identified, where the desired strain is estimated (e.g., by comparing scores associated with respective outcomes of a particular strain) to provide substantially similar outcomes as the desired strain (e.g., for the patient directly providing feedback information).

Accordingly, a patient-centric model is disclosed where the patient expresses honest opinions, findings, beliefs, or experiences of the patient and, when specific results and/or rankings are claimed, such rankings are automatically compared (e.g., by the customer information manager 600) with other patient outcomes (e.g., to filter out patient expressions of untypical patient outcomes) so any posted (e.g., published) outcomes reflect a typical consumer (for example) experience. For example, multivariate statistical analysis of rankings (e.g., for respective outcomes associated with use of a particular strain) in a patient review of a strain product is used to determine whether the patients expressed rankings are similar (e.g., in accordance with principles of standard of deviation) to other reviews for the same and/or similar strain product submitted by other patients.

In an embodiment, a distributed (e.g., using Internet connected components) platform is arranged to survey resources related to particular strains and to select a strain (and or strain provider) in accordance with a desired outcome for a particular user (e.g., human patient). For example, a user interface (such as a GUI, voice-enabled artificial intelligence-based UI, and the like) is arranged to electronically receive user inputs (such as parameters for describing a desired outcome). A strain engine is arranged to process the received user inputs in accordance with, for example, a geographic context of a patient (including the location of the patient) and/or dispensary, region-specific jurisdictional laws (including regulations, rules, and the like), medical context (e.g., presence of valid prescription, “marijuana card,” and the like), price comparison, delivery method (including selection by location of licensed dispensaries), crowd-sourced user feedback, vendor strain product availability, and the like. The strain engine presents the processed user inputs to the user in accordance with a degree of matching (e.g., ranked in order of increasing similarity to values of the user-supplied parameters, popularity, paid advertising, and selectively weighted combinations thereof).

In an embodiment, the strain engine can electronically receive information from a vendor (e.g., associated with a dispensary from which the desired strain can be procured and/or otherwise dispensed). For example, the vendor provides a catalog of products (e.g., menu, inventory, product list, and the like) of strains products (e.g., in any form having ingredients derived—or copied from—cannabis-related plants) that are potentially available from the vendor. The strain engine filters the vendor catalog (e.g., “menu”) to select strains more closely matching the received user inputs (e.g., for selected outcomes for a particular patient). The strain engine provides a “vendor strain score” for each of the strains to help the user select alternative, yet closely matching strains (e.g., that are likely to produce the selected outcomes). The unified score can be an individual number, and/or a number(s) and/or letter(s) graphically presented to convey magnitude in various dimensions displayed in chronologically descending order (such as size for cost, color for closeness in desired outcomes, outline shape for medically licensed, location in a map for geographical location of a vendor, and the like).

In an embodiment, the strain engine tracks the demand of customers in an area. For example, customers in a certain zip code of an urban area search for a particular strain but the search results indicate that no vendor is found in—or close to—the area defined by the zip code. Such data, for example, can be sold to the vendors (or a preferred vendor) in order to meet the demand indicated by indication of the lack of supply of the searched-for strain. As described below, areas, locations, and/or distances relative to a customer-related geographical coordinate—including sets of such coordinates for defining an area—can be expressed in terms of convenience to a particular customer (such as a residence, a route traveled, zip code, distance to a present location, and the like).

For example, the strain engine is arranged to track strain product search requests from a particular customer and to store each and every strain product search requests the particular customer as well as from other customers, where the particular customer and each of the other customers are associated with a respective (e.g., for each) customer location geographically concentrated with respect to (e.g., near to) a vendor dispensary location. The strain engine is arranged to search the stored strain requests to identify most highly searched-for (e.g., by the customers) strain products and to an indication of the identified most highly searched-for strain products to the vendor dispensary (e.g., so the vendor is aware of a geographic demand for particular or similar strain for purposes of marketing and stocking).

Accordingly, a customer location can include one or more of an address, current location, predicted location, customer-selected location, and the like. The predicted location for a customer at a selected time can be estimated, for example, by tracing actual current customer locations at various time points. Such traceable customer locations include lists of locations and associated time recorded and/or reported current customer locations by mobile apps, current customer locations stored when receiving strain search requests, dispensary check-in times by the customer where the dispensary is associated with a location, and the like.

In an embodiment, the strain engine provides a cannabis recommendation in accordance with the vendor strain scores (e.g., provided by the vendor) and a particular user's (e.g., customer's) profile. The user's profile includes, for example, personal information (e.g., address, age, current location, and the like) as well various data points related to treatment such as positive effects, medical benefits, preferences, desired outcomes, negative side effects, location, and affordability. A user (e.g., patient, the customer, user, and the like) score is determined in accordance with selectable inputs. For example, the user is able to select parameters for determining a user score for the user. If the user is primarily concerned about health benefits, the user can select (and assign a priority) to attributes relative to certain health benefits, such that the user score indicates the particular selected health benefits. When a user selects multiple categories such as positive effects, medical benefits, and negative effects, the user score is indicative of the three selected sets of characteristics so the user score provides an indication of the desired characteristics to the strain engine for determining a strain recommendation.

In an embodiment, a strain finder receives the determined strain recommendation to determine the prices and location of multiple dispensaries which carry the determined strain (as well as strains similar to the determined strain). The pricing and geographical location of the sources (e.g., vendors, vendor dispensaries, vendor-related delivery services) of the determined strain are geospatially presented on a displayed map, as well as listed with relevant sorting options (such as by price, distance to user, ratings by other users, whether they are open or closed at the time of the search, and the like) where icons and data are listed and layered over geographic data (e.g., map) in accordance with a user's relative proximity to the sources. A user can choose to limit the range of the search by specifying a radius limit, which selectively localizes searching for available (e.g., located) optimal strains (e.g., where optimal strains have a vendor strain scores closely indicative of producing selected patient outcomes as determined from a patient input on a search screen and/or the patient's profile).

In accordance with embodiments of the present disclosure, a user can in real time locate a determined (e.g., desired strain), select substitute strains, and discover new (e.g., but similar to the determined a strain) strains with relative ease. Further, a user is able to operate an Internet-connected device to quickly discern locations (and existence of) desired strains selected in accordance with the user's expected outcomes (e.g., in cost, distance, biological effects, legality, and the like).

In an embodiment, a user interface (e.g., a GUI) of the disclosed strain finder receives user input concerning (e.g., negative) outcomes that the user intends to avoid (e.g., drowsiness, which might be a desired outcome for another user). The strain engine is arranged to more heavily deprecate unified scores (e.g., disproportionately to algorithms for promoting strains having desired outcomes) so that, for example, extra care can be taken to avoid outcomes that are to be avoided.

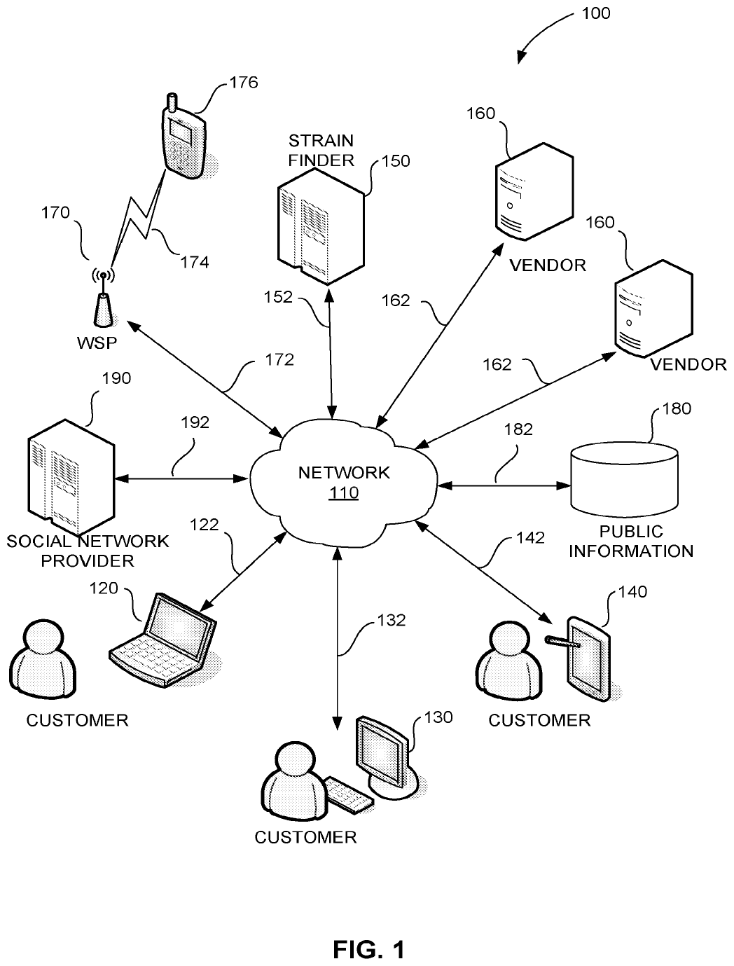

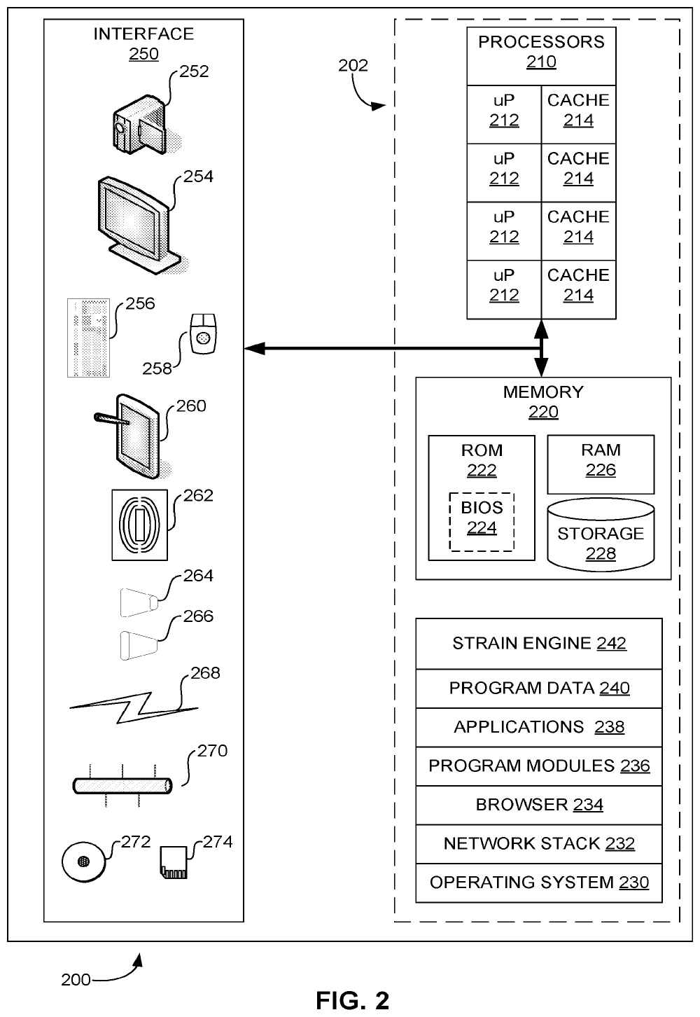

FIG. 1 is a network diagram showing a network suitable for practicing aspects of selection and/or finding of desired cultivated strains in accordance with exemplary embodiments of the disclosure. Network system 100 includes customers (e.g., users, patients, care providers, and the like) 120, 130, and 140 (for example), strain finder 150, social network provider 190, vendor(s) 160, wireless services (e.g., cellular) provider 170, and public information 180. Customers 120, 130, and 140 access and communicate with network 110 using communication links 122, 132, and 142 respectively. Each of the customers 120, 130, and 140 can be (or internally provide functions of) the (illustrative) computing device 200 discussed below with reference to FIG. 2.

Network 110 typically includes a publically accessible network such as the internet, but other networks (including private networks) can be used. Thus, network 110 is typically a collection of networks (and gateways) that typically use a TCP/IP suite of protocols for packet-based communications. The internet typically employs high-speed data communication lines between major nodes or host computers, but even bandwidth between the major nodes is subject to degradation through satellite outages, hardware faults, denial of service attacks, oversubscription of services, and the like. The network connections are shown for the purpose of illustration, and other ways of establishing a communications link between computers (such as using firewalls, as discussed below) can be used.

Customers 120, 130, and 140 access the network 110 for accessing providers of services such as strain finder 150, vendor 160, wireless service provider 170, and public information 180, and social network provider 190. The customers 120, 130, and 140 can access the strain finder 150 to identify a desired strain provided (e.g., sold, offered, dispensed, and the like) by a convenient (e.g. based upon location, price, other user reviews, and similarity to an optimum strain) vendor. The customers are able to access the strain finder 150 using any suitable connected device (e.g., mobile application, Internet-connected television, laptop, phone, computer desktop, networked automobile, Alexa, and the like) or disconnected device (e.g., a computer in which a searchable database has been downloaded, a digital e-book, and the like).

The customer 120 is arranged to provide user-supplied information to the strain finder 150. The strain finder 150 searches stored information to locate highly pertinent information for selecting a product associated with (e.g., made using components of and/or including) an optimal strain. The strain finder 150 is arranged to compare a customer location to determine one or more convenient locations for dispensing a optimal strain. The strain finder 150 is arranged to send an inventory of a selected vendor to the customer 120 such that, for example, the user can select a vendor of the product associated with an optimal strain to reserve or buy the product associated with the desired strain. A third party API (application programmers interface) can be instantiated on the strain finder 150 or the customer 120 so that, for example, customer searches, preferences, and orders can be automatically forwarded to a selected vendor.

The strain finder 150 accesses network 110 via communication link 152, whereas vendors 160 accesses network 110 via a respective communication link 162. Vendor 160 can be a services provider such as online store and/or a physical “brick and mortar” establishment. Wireless service provider 170 accesses network 110 via communication link 172 and provides, for example, further connectivity to cellular devices 176 via a cellular network 174. Public information 180 is coupled to network 110 via communication link 182 and provides, for example, publically accessible information (including services) for tax rates (e.g., for a geographical area and/or jurisdiction), laws (including rules, regulations, and procedures) for using (include sale, possession, transportation, dosing, and the like) controlled substances, licensing, health care providers, confidential information (e.g., electronic medical records and/or electronic health summaries for which a client or government entity has granted access), and the like. The social network provider 190 is coupled to the network 110 via communication link 192 and is arranged to facilitate communication (including promoting services) of the strain finder 150 with respect to customers 120, 130 and 140. The actual data processing systems of network system 100 may include additional servers, clients, and other devices not illustrated. Each of the strain finder 150, vendors 160, wireless service provider 170, data storage provider 180, and social network provider 190 can be (or internally provide functions of) the (illustrative) computing device 200 discussed below with reference to FIG. 2.

FIG. 2 shows an illustrative computing device 200 in accordance with exemplary embodiments of the disclosure. For example, the computing device 200 includes a processing system 202 that is arranged to perform specific tasks in response to applications 238 and program data 240. Processing system 202 is often incorporated into a computing device such as a mobile device, a personal digital assistant, a personal computer, a dedicated web-enabled appliance, a kiosk terminal, automotive electronics, or any other type of networked electronic system or subsystem.

The processing system 202 includes processors 210 and memory 220. Processors 210 may include one or more microprocessor (uP) cores 212, each of which is optionally coupled to a local cache 214. Memory 220 includes a ROM (read-only memory) 222, RAM (random-access memory) 226, and storage 228 (such as a “hard” disk). ROM 222 optionally includes BIOS (basic input/output system) 224, which typically includes low-level firmware-based drivers for accessing, for example, low-level, hardware-based elements of computing device 200.

Memory 220 includes instruction and data for executing (software) applications 238 (for example), that when executed by processing system 202, perform any suitable function associated with the computing device 200. For example, the processing system 202 executes software (including firmware) and data components such as operating system 230, network stack 232, browser 234, program modules 236, applications 238, program data 240, and machine-executable instructions for converting memory 220 and processors 210 into a special-purpose strain engine (e.g., tangible machine) 242 for performing strain finder functions.

Processing system 202 is accessible to users and non-local components using interface 250. Interface 250 provides a user interface that is typically arranged to provide output to and receive input from the user during the execution of the software applications 238. The output to the user is provided by devices such as the display 254 (including indicator lights and image projectors), a speaker 264, vibrations 262, and the like. The input from the user is received using keyboard 256, mouse (and/or trackball) 258, touch/stylus screen 260, audio input 266 and/or video input 252. Other devices can be used such as keypads, switches, proximity detectors, and the like. The interface 250 includes (and/or can be combined with) various metaphors such as augmented reality, virtual reality (including virtual assistants in a headset), voice-controlled artificial intelligence, (including Amazon Alexa, Ski, Cortana, and the like), bots (including Slack Bot, Messenger Bots, and the like), SMS (short message system) text messages, and other such methods of (e.g., personalized) electronic communication.

The interface 250 is also arranged to transmit communications to and from other computers across a network. Wireless link 268 permits communications using a modulated optical and/or electromagnetic carrier (such as cellular telephone communications). Cabled link 270 permits communications over a wired and/or optical link (such as optical Ethernet and/or Ethernet). The wireless link 268 and cabled link 270 are optionally employed between other network-enabled devices to establish wide-area networks, local-area networks, private networks, and the like. For example, geographic coordinates can be derived via local processing of electronic signals from microelectronic accelerometers and/or from cellular towers, satellites, and/or wide-area augmentation navigation systems earthbound and/or orbital (GPS, GLONASS, and the like). Additionally, non-transitory, tangible media such as disk 272 or “flash” ROM 274 (and the like) are used to store data and instructions and are read from and/or written to by interface 250 in the course of processing inputs and outputs of the strain finder, for example.

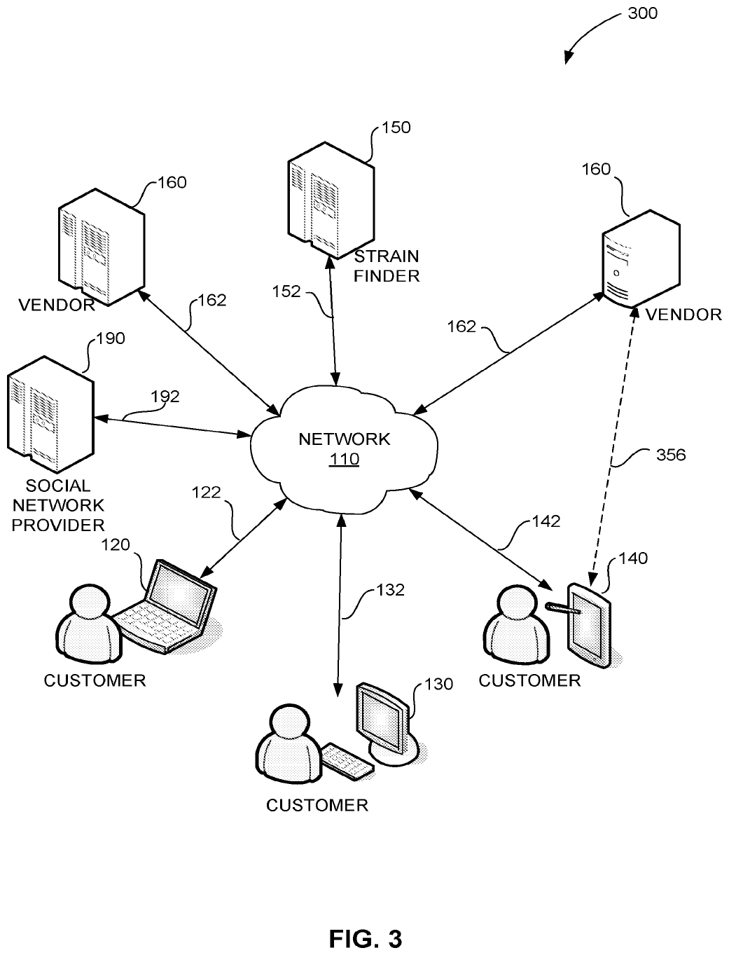

FIG. 3 is a network diagram showing a network for selection and/or finding of desired cultivated strains in accordance with exemplary embodiments of the disclosure. Network system 300 includes customers 120, 130, and 140, strain finder 150, vendors 160, and social network provider 190 as discussed above. The strain finder 150 is arranged to receive information from and provide content (such as information and/or applications, and the like) to the customer 120 via network 110.

The content and services are generally provided in the form of communications such as webpages, where the webpages (and other communications) are GUIs including listings of products, goods, or services that are offered by the vendor 160. The (e.g., browser of) customer 120 is arranged to receive web page from the strain finder 150 for entering customer-specific parameters for locating an optimal strain for the customer 120). The strain engine 242 of the strain finder 150 is arranged to receive the customer-specific parameters and to select strains for proving outcomes (e.g., results) more closely matching strains. The selected strains can be, for example, presented as a ranked list of strains ordered in accordance with a unified score where the ranked list is presented to the customer as a list (e.g., showing inventory of a provider geographically near the customer and/or icons on a map (e.g., showing locations of vendors for providing a desired strain).

A “backchannel” 356 is optionally arranged between the customer 120 and vendor 160. The backchannel 356 can be a phone call, text message, email, and the like for ordering/reserving a particular strain to be dispensed. The backchannel 356 is arranged to, for example, permit (e.g., anonymous) communications between a customer 120 and a vendor 160 if any of (or portion of) network 110, communication link 162, and communication link 162 fails.

The social network provider 190 is a server (or a set of servers that are presented as a single server or a “virtual” server for processing requests). The customers 120, 130, and 140 are typically clients with respect to the server (e.g., strain finder 150). The customers 120, 130, and 140 are, for example, personal computers or network computers being operated by customers of one of the vendors 160. The social network provider provides a “wall” (e.g., graphical electronic bulletin board) under the control of (e.g., administrators of) the strain finder 150 so that, for example, goods and services of the strain finder 150 are promoted for the benefit of the customers 120, 130, and 140.

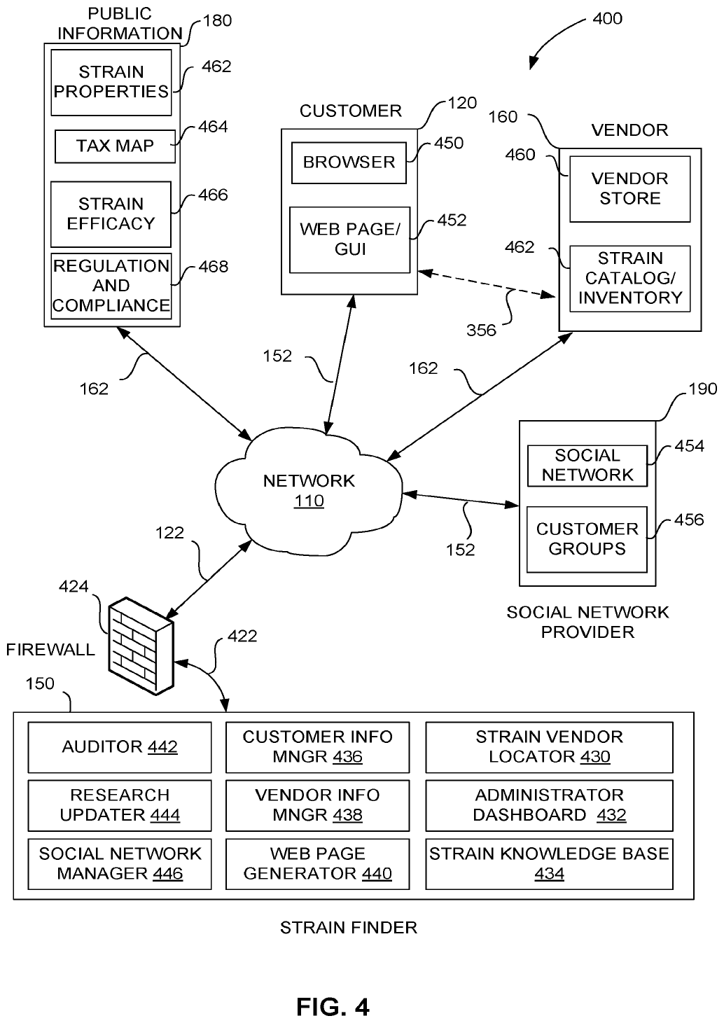

FIG. 4 is a logic diagram showing a system for selection and/or finding of desired cultivated strains in accordance with exemplary embodiments of the disclosure. The network system 400 includes, for example: customer 120, strain finder 150, vendor(s) 160, public information 180, and social network providers 190.

The strain finder 150 is arranged to (more securely) communicate with network 110 using communication link 122, firewall 424, and communication link 422 so that, for example, communications between the strain finder 150 and (e.g., any of) the customer 120, vendor 160, public information 180, and the social network provider 190 is robust and secure.

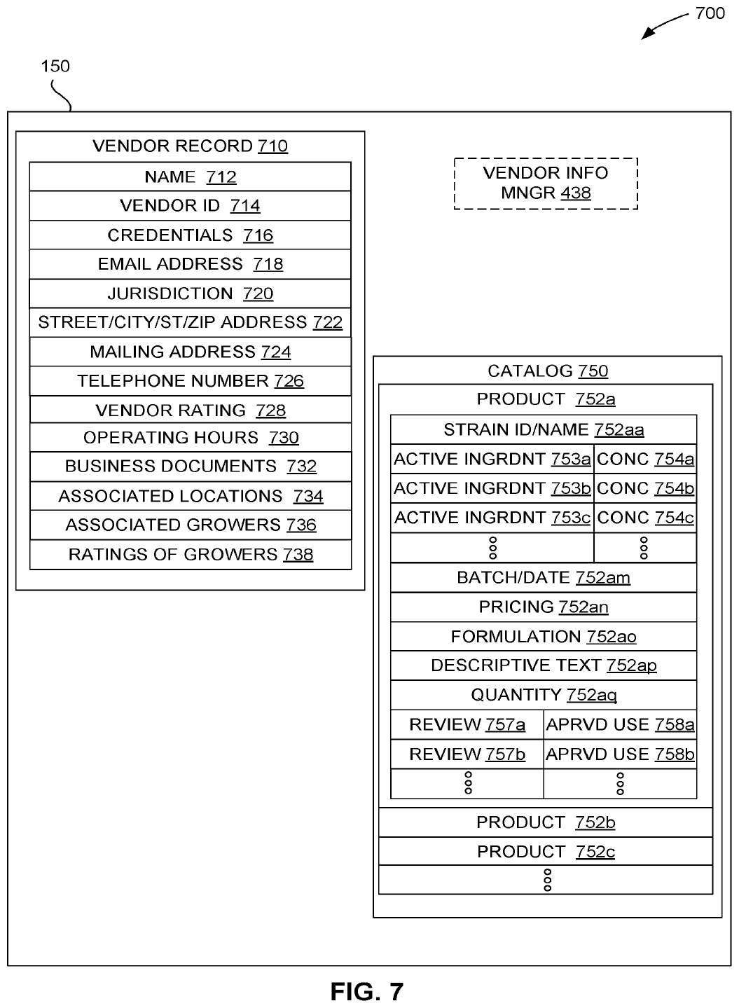

Vendor 160 includes a vendor store 460 as well as a strain catalog and/or inventory 462 (e.g., where strain catalog is an electronic listing off particular strains, strain products, and related quantities, concentrations, formulations, and the like as discussed below with reference to FIG. 7). The vendor store 460 is typically an electronic portal of the physical (e.g., brick and mortar) store for vending and/or dispensing physical strain-related products. The vendor 160 typically uses the vendor information manager 438 (as discussed below with reference to FIG. 7) to upload and update inventory information to the strain finder 150.

The customer 120 (e.g., the computer operated by a customer) typically includes a browser 450. For example, the browser 450 includes a browser such as Chrome, Firefox, Internet Explorer, Edge and the like. Accordingly, the communications typically include webpages written in a markup language, although other formats can be used. The markup language is used to convey the structure and functionality of a graphical user interface 452 presented using a web page metaphor. The browser 450 is also arranged to store search histories (including “favorite” web addresses) and cookies served to the browser 450 by the strain finder 150 and/or the vendor 160. Although the disclosed embodiment includes a browser 450 as a user interface (UI), other UIs are possible, including technologies such as virtual and/or augmented realities, bots, SMS, voice-enabled AI, and the like.

The browser 450 is also arranged to browse the social network 454 so the user of customer 120 can browse the social network “wall” (e.g., “page” and/or electronic bulletin board) of the strain finder 150 administrators as posted on the social network 190. Social network providers 190 include (e.g., different) social networks 454 associated with customer groups 456 typically executing on massively paralleled servers. The social network manager 446 of the strain finder 150 is arranged to monitor and update communications of the social network 454.

The browser 450 is arranged to conduct communications between the customer 120 the strain finder 150 and/or conduct communications between the customer 120 and vendor store 460. For example, the browser 450 is arranged to display web pages served by either (or both) the web page generator 440 of the strain finder 150 and the vendor 160.

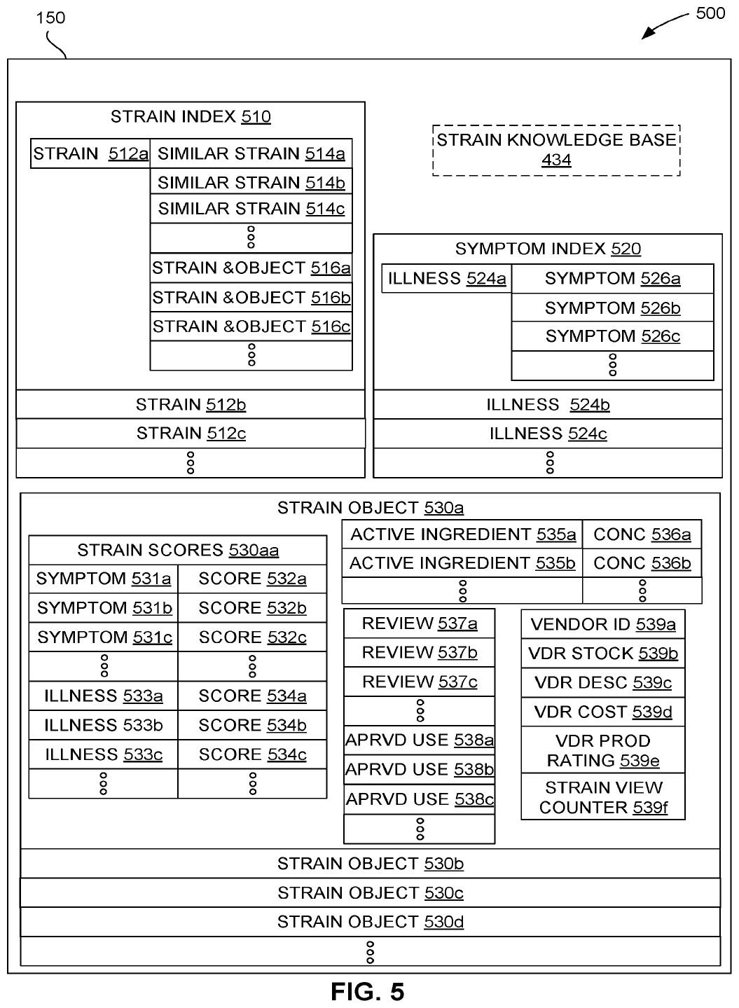

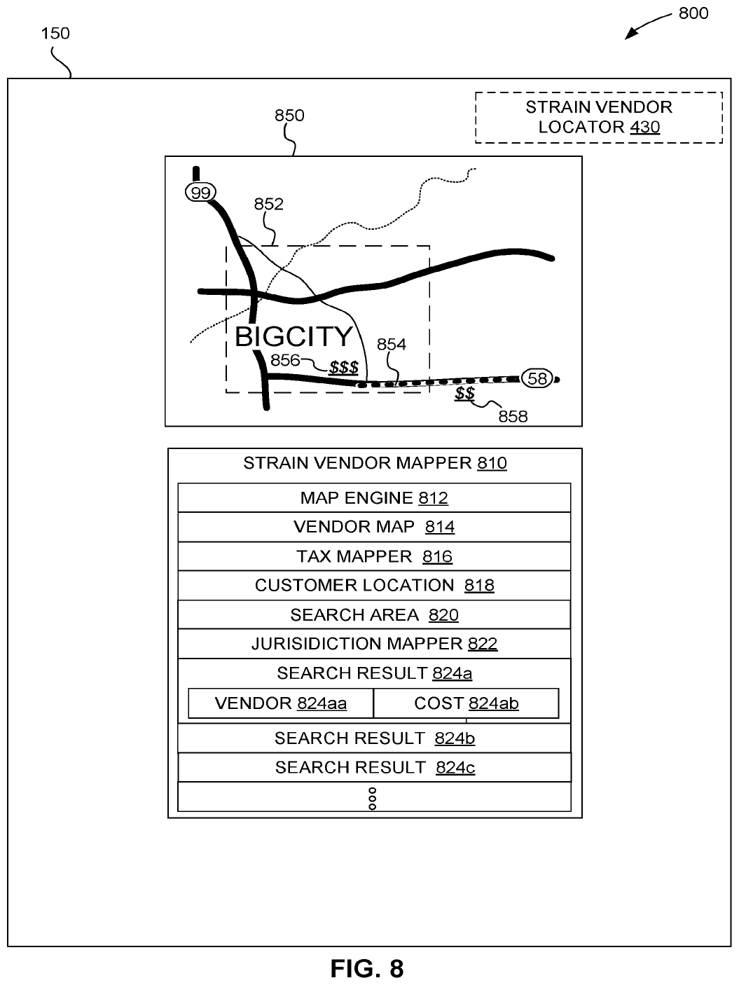

As discussed below with reference to FIG. 5, the customer 120 is arranged to input user information such as, search parameters, reviews and the like to the strain finder 150 (via the customer information manager 436). The strain (e.g., strain product) finder locator 430 (discussed below with reference to FIG. 8) is arranged to compare a customer location (e.g., a fixed address or an instant location of the customer) with locations for dispensing a desired strain. In response, the strain finder locator 430 the strain finder 150 provides highly related (e.g., relevant) information to the customer 120 for selecting a vendor of an optimal strain. When the user selects a vendor, the strain finder 150 displays the inventory of the selected vendor in accordance the vendor's selected strain scores and stored user information (including user scores for scoring desired outcomes). The inventory of the selected vendor is ranked using the vendor's selected strain scores and the stored user information. Accordingly, the inventory of the selected vendor is displayed in a ranked order (and is selected in accordance with proximity of the user to a source of the desired strain).

The customer 120 is also arranged to input user information such as user reviews and ratings including user-supplied outcomes associated with (e.g., personal use of) a particular strain. The customer information manager 436 stores the input information in association with the customer 120 so that, for example, the customer's rating of the effects of a particular strain are used to rank search results when searching for an optimum strain for the customer. The user rating, reviews, and optionally vendor-provided information related to the effects of particular strains are also consumed by the strain knowledge base 434 (described below with reference to FIG. 5), which is arranged to associate particular strains with desired (e.g., intended) outcomes (e.g., efficacy with regards to various symptoms and illnesses, mood enhancement, elevation of well-being, and the like), associate with vendors selling a particular strain, and associate other information unique to a particular strain.

The public information 180 is a publically accessible information data source generally viewed to be reliable. For example, the public information 180 includes strain properties 462, tax map 464, strain efficacy 466, and regulation and compliance 468 information. The strain properties 462 includes research and information about particular strains (including a list of active ingredients, concentrations thereof, and other biological aspects) and is crawled by the research updater 444 to flag recent developments for the benefit of administrators of the strain finder 150 (e.g., to manually or automatically update the below-discussed strain objects 530). The tax map 464 includes information of the tax rates of various jurisdictions (including an aggregate tax amount associated with a jurisdictions overlapping a particular geographical coordinate such that, for example, a tax amount can be determined in response to a determination of the presence of prescription for a particular customer and legality of the jurisdiction). For example, a prescription can lessen or eliminate taxes (including legally mandated associated surcharges) otherwise collected in response to selling/dispensing cannabis. The strain efficacy 466 includes effects and scores (e.g., 1-10), ratings (e.g., littlest-biggest) and/or rankings (e.g., “a” is higher than “b”) for a particular strains (e.g., for particular users and/or classes of particular users). The regulation and compliance 468 information includes information regarding laws and regulations concerning cultivation, manufacture, sale, possession, use, and distribution of regulated substances. The public information 180 is used to update the strain finder 150 automatically (e.g. via the auditor 442 when such information is in a known location and/or is easily parsable) or manually (by an administrator using the administrator dashboard 432 of the strain finder 150). Accordingly, the strain finder 150 is arranged to consume the publically available (e.g., externally sourced) information to update internal records of strain ingredients (e.g., intrinsic qualities of a strain product) and efficacies, determine accurate tax costs associated with a geographical location for determining sources from which to dispense recommended (e.g., by the strain finder 150) strain products, and help ensure compliance with locally applicable (e.g., with respect to a location associated with a customer) laws, rules, and regulations.

FIG. 5 is a logic diagram of a strain knowledge base 500 in accordance with embodiments of the present disclosure. The strain knowledge base 500 is a knowledgebase such as the strain knowledge base 434. The strain knowledge base 500 includes the strain index 510, the symptom index 520, and a list of strain objects 530.

The strain index 510 is arranged to associate similar strains with a particular strain, where the similar strains, for example, have similar scores with respect to desired outcomes. The strain index 510 also maintains pointers (e.g., “&object”) to each strain object 530 in the list of strain objects so, for example, information about a strain (e.g., score, cost, where stock is available, vendor rating, and the like) can be retrieved in (e.g., an indexed) association with a strained name (or unique identifier). Accordingly the strain index 510 includes strain identifiers (e.g., 512a, 512b, and 512c) wherein each strain is (e.g., potentially) associated with similar strains (e.g., 514a, 514b, 514c) and strain objects (e.g., 530a, 530b, 530c, 530d). Accordingly, each strain identifier 512 can be associated with, for example, differences in scores and costs and depending on where a particular strain was cultivated and/or purchased. The strain knowledge base 500, for example, selects optimum strains for a particular user at a particular (or estimated) time at (or associated with) a particular user location in accordance with the location and optionally cost of a selected strain in-stock at a particular vendor dispensary.

The symptom index 520 is arranged to associate various selected outcomes and/or symptoms (e.g., 526a, 526b, 526c) with a particular illness (e.g., 524a). The symptom index 520 includes a list association with a list of symptoms for various illnesses (e.g., 524a, 524b, 524c). Accordingly, an optimum strain can be searched for using illnesses and/or symptoms as search terms used in scoring rankings of potential optimum strains. For example, licensed physicians can each provide a numerical value (scored ranking) for using a selected strain for treating a particular symptom of a particular illness, where the numerical value indicates a physician's estimation of the efficacy for treating the specific symptom on a typical patient. The plurality of provided numerical values can be normalized (e.g., averaged, with outliers rejected) such that the symptom index includes normalized scores/rankings for a typical efficacy of a particular strain for treating a specific symptom of (and optionally selected) illness. The scores/rankings can be rated on a scale of 1-5, with optionally providing negative rankings when use of a certain strain would be contraindicated in treating a certain illness and/or a particular symptom of the certain illness (e.g., so that normalized scores would not indicate the use of a certain strain in treating a certain illness or a particular symptom of the certain illness). (The licensed physician can access and store information)

Each strain object 530 (e.g., 530a, 530b, 530c, 530d) includes a list of strain scores (e.g., 530aa) where each symptom (e.g., 531a, 531b, 531c) is associated with a respective score (e.g., 532a, 532b, 532c). The term “symptom” can include the meaning of “effect,” “desired effect,” “side-effect,” “outcome,” “result,” “benefit,” and the like. When a particular symptom is to be treated (for example), the score is treated positively for ranking purposes and when the particular system (such as a side effect) is to be avoided (for example), the score is treated negatively for purposes of selecting a desired strain. Accordingly, the strain knowledge base 500 is arranged to generate a match score in response to a set of chosen effects, benefits, and (e.g., adverse) side effects (e.g., collectively, “symptoms”). Such symptoms and/or effects, for example, are represented by labels like “happy” or “creative,” benefits are represented by things like “pain relief” or “energetic,” and side effects by “anxiety” or “dry mouth.” It is apparent that the descriptive labels for the symptoms can overlap (in which case the scoring can tend to likewise be similar in accordance with the degree of overlap). While the labels used across the different databases may be the same (or synonyms), the values of the scores of each individual strain object 530 differ from the individual scores given by various users (e.g., patients, “ordinary” customers, experts, licensed physicians, vendors, and the like).

Each strain object 530 (e.g., 530a, 530b, 530c, 530d) includes a list of strain scores (e.g., 530aa) where each illness (e.g., 533a, 533b, 533c) is associated with a respective score (e.g., 534a, 534b, 534c). Accordingly, the strain knowledge base 500 includes a search engine and/or algorithm arranged to generate a match score in response to an identification of a particular illness. For example, the match score is indicative of optimal or near-optimal strains for treating a specific illness (such as glaucoma) so that strains having relatively high scores are selectable as an optimal strain (or near-optimal strain) for treating a particular illness. The optimal strain (e.g., optimal strain product) is used (e.g., as a search “term”) by the strain knowledge base to search for vendors having dispensaries (e.g., stocking the optimal or near-optimal strain) in an area (or “location”) geographically related (e.g., “near”) to a patient searching for an optimal strain (or near-optimal strain).

Each strain object 530 includes a list of active ingredients (e.g., 535a, 535b) and associated concentrations (536a, 536b). For example, a first active ingredient can be delta-g-tetrahydrocannabinol (THC) and the second active ingredient can be (e.g., a class of) cannabidiol(s) (CBD). Other active ingredients, such as cannabinol (CBN) or other classes or instances of active compounds of cannabis products, can be likewise be listed and searched for. The concentrations of the active ingredients can vary as a function of a particular source (e.g., vendor, grower, growing conditions, and combinations thereof): accordingly, the concentrations 536 can be supplied by a lab or the vendor of the particular strain the supplied by the vendor (see FIG. 7, for example). The concentrations can be used to avoid dispensing product above regulatory limits (e.g., based on age, location of patient and/or dispensary, existence of a prescription, existence of a medical marijuana card, and the like) and can be used to assess a proper level of taxation based on the concentrations of the strain (and/or product including regulated strain by-products) and location of the strain vendor and/or dispensary. The amount of the concentration of a particular active ingredient can be specified (e.g., as a range associated with a named ingredient) in a search so that (for example) the customer can locate strain products having a selected active ingredient in concentrations within a certain range. A third-party API can be used to (e.g., automatically) populate lists of active ingredients for a known strain.

Each strain object 530 includes a list of reviews (e.g., 537a, 537b, 537c), where the reviews (or links to the reviews) can be supplied by the customer and/or social network user. The reviews can be evaluated by rule bases and/or artificial intelligence- (AI-) based monitors for culling outlier reviews and flagging suspicious reviews for an administrator/moderator. The reviews can be “surfaced” in response to a link displayed to a customer browsing information related to a particular strain supplied by a particular vendor (e.g., “strain object,” “strain batch,” and the like). Accordingly, strains having the same name, but different sources, can be independently reviewed and searched for (so, for example, vendors of either good and bad products having the same strain name can be identified).

Each strain object 530 includes a list of approved uses (e.g., 538a, to 538b, 538c), where the list of approved uses is typically provided by the vendor so that a potential customer can use to-be provided products in accordance with an intended use, and, to the contrary, avoid suggesting (and/or incurring liability therefor) an unintended or prohibited use of a strain product (e.g., identified by a selected strain object).

Each strain object 530 is associated with a vendor ID 539a, a vendor stock number 539b, the vendor description 539c, a vendor cost 539d, a product rating of the product supplied by a particular vendor and/or dispensary (e.g., grades of “A” through “F” or number of stars) 539e, and a strain view counter 539f. For example, the vendor ID 539a identifies the particular vendor and the vendor store number 539b identifies a particular batch and/or formulation (sativa, indica, hybrid) related to the (e.g., named) strain. When a new batch of a named strain is introduced as a new strain object 530, values from a previous batch of the same named strain can be (e.g., provisionally) populated into various fields of the strain of object 530 by default (e.g., by principles of inheritance). The vendor description 539c and the vendor cost 539d are provided by the vendor (as described below with reference to FIG. 7). The vendor products rating 539e can be a number (e.g., 5-stars) grade determined in response to any of aggregated user-ranked scores of the efficacy of strains provided by the vendor, (e.g., unexpected) negative side effects of strains provided by the vendor, consensus of user-supplied and social network reviews about the vendor, administrator opinions about the vendor, and the like. The strain view counter 539f is an indication of a particular strain's popularity, where the strain view counter is a count of the number (or frequency) the particular strain is accessed by the knowledgebase 530 (e.g., as accessed by customers or ‘recommended,’), reviewed (number of reviews as well as the aggregate quality of the reviews). The value displayed by the strain view counter 539f can be determined in response to the number of qualified (e.g., a maximum one impression per IP address) strain views (or other action implicating a selected strain) per month (or, for example, using other such dynamically selected rules applied by a rule base). The value of the strain view counter 539f (which is associated with a particular strain product) can be propagated to the related vendor for planning and/or stocking purposes, for example.

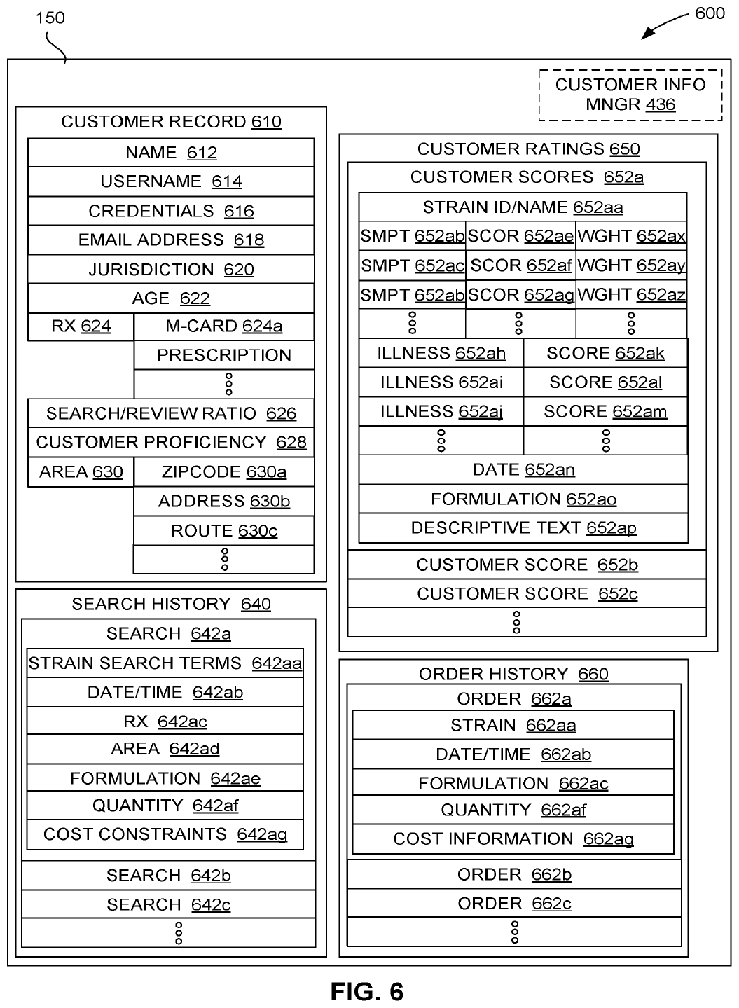

FIG. 6 is a logic diagram of a customer information manager 600 in accordance with embodiments of the present disclosure. The customer information manager 600 is a manager such as the customer information manager 436. The customer information manager 600 includes one or more customer records 610, a search history 640, customer ratings 650, and an order history 660. The customer information manager 600 is arranged to present a user interface to the customer containing pre-populated fields (e.g., when known or reasonably guessed—such as a previously used value or normalized values based on other users having the same or similar characteristics in their profile pages) and/or a list of symptoms/effects/outcomes having “sliders” so the customer can quickly enter information for their review.

Further, the customer has the option of editing the strain knowledge base (e.g., 500) ranking associated with a particular strain recommended by the strain knowledge base and evaluated by the customer. For example, a user can “customize” the rankings presented by the strain knowledge base by moving sliders preset to the presented rankings such that a customized ranking is generated by (e.g., and for) the customer. Accordingly, the customer can readily and easily provide customer rankings (e.g., of outcomes for a particular strain) or text reviews for customizing presentation of strain searching results. The strain knowledge base 500 evaluates (e.g., lexically analyzes) customer edits to the strain knowledge base rankings and automatically adjusts associated (e.g., similar strains having relatively similar percentages of active ingredients, strains having the same name but obtained from different vendors and/or growers, similar strains obtained from different geographical sources and/or seasons, and any strains having strain object-inherited fields) scores supplied by the strain knowledge base in accordance with a weighting function determined in response to a customer-related “degree of importance” of a particular strain knowledge base ranking.

The strain knowledge base (e.g., virtually) adjusts the value strain knowledge base rankings in accordance with the target audience of the strain knowledge base rankings. For example, user-supplied edits to the strain knowledge base-supplied rankings are (re-) presented to the particular user supplying the edits, whereas the user-supplied edits to the strain knowledge base-supplied ranking (e.g., for a respective outcome) are (for example) averaged with other such user-supplied edits from users and the averaged rankings are used to adjust the strain knowledge base rankings (e.g., for a particular strain supplied by a particular vendor, if a vendor is associated with the user ranking by the user supplying the ranking) presented to (almost) all users. Accordingly, the strain knowledge base 500 is arranged to automatically adjust for differences in the utility of a specific strains (e.g., and related products) resulting from cultivation and use in various geographical locations (or other factors) and obtain from various vendors.

The customer record 610 is arranged to store customer-related information for a variety of purposes. For example the customer record 610 includes fields for a customer name 612, a user (e.g., screen) name 614, credentials 616 (for login and/or authentication), an e-mail address 618, jurisdiction (e.g., state) 620, age 622, prescription (RX) 624, M-Card (medical marijuana card) 624a and the associated expiration date, prescription 624b (and/or other regulatory compliance document), search-review ratio (numbers of searches per review written by the customer) 626, customer proficiency (based on usage, occupation, known experience, verified credentials including degrees and certifications, numbers of searches and reviews) 628, customer location (e.g., area) 630 (where the area can be expressed by one or more of the zip code 630a, and address 630b, a route 630c, distance from any of the preceding geographical markers, and the like). For example, the customer proficiency 628 is used to identify licensed physicians or other such professionals so that rankings by the licensed physicians can be segregated, aggregated, and averaged to determine a consensus of licensed physicians of (e.g., efficacy for selected outcomes) rankings of typical experiences in treating various patients.

The search history 640 includes a list of searches (e.g., 642a, 642b, 642c) performed by the customer. Each search 642 is recorded by the customer information manager 600 in response to the customer performing a search. Each search includes strain search terms 642aa, date and time 642ab, prescription 642ac, area (e.g., current customer location) 642ad, formulation (e.g., of the strain being searched for) 642ae, quantity (of the formulation being searched for) 642af, and cost constraints (total, price per unit, and the like) 642ag. For example, the data points of the time, day, and place of a particular search are evaluated by the customer information manager 600, to determine geosynchronous behavior of a customer to predict searching behavior and locations so that, for example, incentives (such as coupons) can be given to the customer and/or notice provided to a dispensary that a customer (identified or non-uniquely identified) will likely visit the dispensary for procuring a searched-for strain. Further, mobile apps can track the user's instant location (and/or historical locations) and prompt the user and/or the dispensary with information concerning a searched-for strain.

The customer, for example, can use presented search results to select, order, and/or reserve a quantity of the selected optimal strain for in-person pickup using the search information. The customer can save searches (e.g., by naming a search) so the search can be easily found and repeated. When the customer uses search results from a user session with the strain knowledge base, an indication of relative closeness (e.g., a match percentage) is given for each strain knowledge base-provided optimal strain recommendation relative to the original search criteria that user entered. The indication of relative closeness provides a continuity of user experience changing focus from searching for a type of a strain to searching for a provider of the strain.

The customer rating 650 can be, for example, a customer-generated ranking/rating for a particular product that is optionally associated with a particular strain obtained from a particular vendor (accordingly, a customer can evaluate the particular vendor, the particular strain, or both the particular vendor and the particular strain). In general, the customer rating 650 is generated in response to a user (e.g., dispensary customer) selecting items from a list of potential outcomes such as (e.g., psychological) effects, (e.g., medical) benefits, and negative (e.g., contraindicated and/or to-be-avoided) effects, rating each of the selected items (e.g., on a scale of 1-5), and assessing (e.g., associating) a degree (e.g., of importance and/or strength of effect on the user) for each of the ranked items (e.g., on a scale of 1-5) in accordance with how each of the ranked items impacted the particular user generating the customer rating 650. The degree of importance can be used as a weight to normalize (e.g., functionally weight in accordance with the self-assessed degree of importance to a particular customer) the ratings to as to emphasize positive traits related to the ranked items and to deemphasize (e.g., avoid) negative traits related to the ranked items. The degree of importance for each ranking of outcomes evaluated by an associated customer can also be used to affect the weighting of the associated ranked outcomes by the associated customer upon the aggregation and averaging of user rankings of the rankings of the associated strain in the strain knowledge base that is presented to most users.

The specified rankings can also be provided for a particular method of use (“intended use”). For example, the efficacy (and accordingly, the ranking) for a particular outcome can vary in accordance with the particular method of using the strain product. The user can assign an “intended use” (e.g., method of using/consuming the searched-for strain product) to the ranking, so that, future search requests can discriminate strain products based on the rankings associated particular (e.g., typical) outcomes resulting from a particular method of using of a particular strain product. Accordingly, a user searching for a selected set of outcomes (“energetic,” “creative,” “happy,” and the like) by a particular method (e.g., ingesting, rather than smoking, for example), conveys the particular method in an electronic request (or as a default, such as a default stored in the customer record 610) when searching for an optimal strain.

When receiving a request for optimal strains, the customer information 610 evaluates the request to determine whether the request includes an intended use (and if not, to determine an intended use from a default in the customer record 610), and when an intended use is associated with the request for optimal strains is present, the strain knowledge base searches for strains having the most highly matched rankings of outcomes associated with the particular indicated use. The particular indicated use can be indicated by search request itself, a default specific to the user, and/or a default generated by the system. In an embodiment, where rankings are not provided for a specific use of a ranked product, the rankings for a known use of a particular strain product can be scaled in accordance with scaling factors derived from empirical studies demonstrating the effects of active ingredients on a patient when taken or applied in differing modalities (e.g., methods such as inhalation, ingestion, topical application in oils and/or lotion, and the like).

The customer information manager 600 can also adjust (e.g., weight) the customer-supplied degrees of importance in terms of relative degrees of desirability for each ranked item: for example, psychologically uplifting effects can be more heavily weighted (in general for all users, and in particular to specific customer-supplied information (including medical histories, customer ratings, and statistical analysis of the customer-supplied degrees) and negative effects can be especially (e.g., negatively) weighted by the customer information manager 600 so as exercise an abundance of caution in avoiding undesirable outcomes (notwithstanding customer ratings neutral or adverse with respect to a general consensus of effects of a particular strain). Accordingly, weighting functions are not necessarily linear functions.

As introduced above, the customer information manager 600 is arranged to weight (e.g., for purposes of updating and/or modifying results from the strain knowledge base 500) the reviews of certain “knowledgeable” customers (e.g., who may be recognized by reputation, number and quality of reviews, occupation, education, and the like). For example, the customer information manager 600 can “elevate” (e.g., more heavily weight) the reviews posted by official bud-tenders working at dispensaries, cultivators, chemists (and/or other people and roles that are similarly understood as being “above” the “average” customer). The disclosed weighting functions by quantitatively extending greater value and authority to the review information the knowledgeable customers provide (via the “feedback capability” provided by the customer reviews). The heavier weighting of the reviews and information provided by the “knowledgeable” customers in turn makes “expert opinions” even more valuable or impactful with respect to any weighted scores “surfaced” (e.g., published to the customers or vendors) by the strain knowledge base 500. The use of the knowledgeable customer scores in weighting, for example, the strain knowledge base scores enhance the value of accuracy and applicability of the strain recommendations made by the strain knowledge base. The weighting can be made even more applicable by nuancing the weighting in accordance with the location field of the knowledgeable customers selected for weighting for a particular search, where the location field of the knowledgeable customers indicates a location within a geographically area including the location of the particular customer making the particular search.

The customer information manager 600 is arranged to evaluate (e.g., by comparison of quantitative rankings and lexical analysis of textual) reviews and ratings in in which a customer expresses honest opinions, findings, beliefs, or experiences about selected outcomes of the use of a particular strain. The customer information manager 600 evaluation is compared against other such evaluations for customer reviews of the same (or, for example, similar) strains to determine whether the instant review of the particular strain would reflect a typical consumer experience (e.g., when a quantized multivariate ranking indicates a deviation exceeding, for example, a predetermined threshold from standard of deviation). The evaluation can “flag” (e.g., send a warning signal about and/or quarantine) a questionable review pending further scrutiny, e.g., by an administrator or moderator. The quarantined review/rating is sequestered such that, for example, the review is not “surfaced,” not used in the selection of optimal strains, nor used to determine weights for respective outcomes in reviews/ratings of the same (or similar) strains. The customer information manager is optionally arranged to generate a concordance of each “word” in the reviews such that all (e.g., surfaced) reviews are searchable by any customer using (e.g., arbitrary) search terms any word (even made up, fanciful, and slang) contained in a review can be searched-for.

The customer rating 650 includes a list of customer scores (e.g., 652a, 652b, 652c). Each customer score includes a strain ID name (e.g., 652aa) associated with a list of symptoms/outcomes (e.g., 652ab, 652ac, 652ad) paired with an associated score (e.g., 652ae, 652af, 652ag). The score for each outcome is normalized in an accordance with a respective weight (e.g., 652az, 652ay, 652az), where each weight indicates a degree of importance for a particular customer, and the weight is used (e.g., by the customer information manager 600) to adjust the scores so as to emphasize positive outcomes and to avoid negative outcomes associated with the ranked symptoms/outcomes. The strain ID name can also be associated with a list of illnesses (e.g., 652af, 652ai, 652aj) paired with an associated score (e.g., 652ak, 652al, 652am), which can be optionally weighted as described above. The customer score 652a optionally includes a date 652an (e.g., when the strain product being evaluated was purchase), the formulation reviewed 652ao, and customer-generated descriptive text 652ap (e.g., for conveying a user experience of the strain). The customer rating 650 is queried by the knowledge database 500, for example, to replace and/or modify system or the strain knowledge base ratings for a particular strain/formulation being reviewed. Accordingly each strain ID name (e.g., 652aa) includes a set of ranked outcomes, where each ranked outcome includes a plurality of diverse outcomes, with each such diverse outcome having a ranking associated with the diverse outcome.

The order history 660 includes a list of orders (e.g., 662a, 662b, 662c). Each order 662 includes an identifier of the strain being ordered (e.g., 662aa), the date and time of the order (e.g., 662ab), the formulation of the strain (e.g., 662ac), the quantity of the formulation ordered (e.g., 662af), and cost information (e.g., 662ag). As introduced above, the order history can be evaluated by the customer information manager 600, for example, to post timely (in time and location) notices to the customer and/or post notices to dispensaries for stocking purposes when order histories of various customers indicate (e.g., an increase in) a popularity of a particular strain (and the like).

In various embodiment the customer information manager 600 can track, record, search-for on social media platforms, and recall customer-related attributes such as gender, dispensary check-in history, likes of particular dispensaries, favorites of dispensaries, electronic “likes” of strain and the dispensing dispensary or dispensaries, favorite formulations of strain, and the like. Each customer-related attribute can be used as a weight in providing ranked search results. For example, weighting of selected customer attributes is used to rank and select search results for recommending dispensaries electronically “liked” (such as electronically “liking” a dispensaries page or a selected strain page on a social media platform) by the customer, tracking dosage rates with respect to dispensary check-ins, checking availability of favorite formulations over (e.g., that are in-stock at) favorite dispensaries, and the like.

The strain knowledge base 500 can rate the customer based on the customer's order history for the purpose of, for example, in response to the customer's reviews and rankings (and in particular when the customer directly ranks the strain knowledge base strain recommended to the particular customer, e.g., such that a customer rating for a product can be deprecated when the user has no associated order history). The strain knowledge base 500 also automatically notifies customer when a new strain is added into the knowledge base that matches (e.g., within a threshold for score and a threshold for distance) any saved search criteria (e.g., selected) by the user. The strain knowledge base 500 can also automatically notify a customer when a strain appears locally (e.g., that was previously unavailable) to that user, where the strain matches a set of previously desired attributes (e.g., ranked outcomes) for which an unsuccessful attempt to procure (e.g., buy in-person, order, have delivered, place on hold, and the like) a locally supplied matching strain was made. The order can be verified by comparing vendor records with the customer's order history. Anonymity can be preserved by the use of one-way digital signatures (e.g., hashes) for example.

FIG. 7 is a logic diagram of a vendor information manager 700 in accordance with embodiments of the present disclosure. The vendor information manager 700 is a manager such as the vendor information manager 438. The vendor information manager 700 includes a vendor record 710 and a catalog (e.g., inventory) 750. The vendor information manager 700 is arranged to present a user interface to the vendor containing (for example) pre-populated fields (e.g., when known or reasonably guessed—such as a previously used value) and/or a list of symptoms/effects having “sliders” so the vendor can quickly enter information for populating their business information and/or their product offerings.

The vendor record 710 is arranged to store vendor-related information for a variety of purposes. For example the vendor record 710 includes fields for a vendor name 712, a vendor ID 714, credentials 716 (for login and/or authentication), an e-mail address 718, jurisdiction (e.g., state) 720, physical address (e.g., street, city, state, zip code, and the like) 722, mailing address 724, telephone number 726, vendor rating 728, and operating hours 730.

The business documents 732 can be used to determine the (e.g., legal and/or permissible) types of transaction offered by the vendor. Each vendor may have one or more associated locations (e.g., dispensaries and locations) 734. Each associated location 734 can be entered as an individual vendor record 710 such that, for example, performance of each associated location 734 can be tracked. A relationship between a dispensary and each of the associated growers 736 (e.g., name of each grower/farmer/cultivator/botanist and/or middleman). The ratings of growers 738 tracks ratings of each associated grower over time. Other vendor-related information can be stored in various fields and/or list elements as needed.

The vendor catalog 750 includes lists of products (e.g., 752a, 752b, 752c), where each product is a particular product associated with a particular strain obtained from the particular vendor. Each product listing includes a strain ID name (e.g., 752aa) and a list of active ingredients (e.g., 753a, 753b, 753c) associated with a paired list of respective concentrations (e.g., 754a, 754b, 754c). The strain ID name can also be associated with a batch number and/or freshness/expiration date 752am (where, for example, the concentrations of active ingredients can be tracked from batch to batch and/or a decrease of the active ingredients can be estimated over time), pricing information 752an, strain formulation 752ao, vendor-generated descriptive text 752ap, and quantity of the formulated strain on hand 752aq. The vendor catalog further includes a list of customer-supplied reviews (e.g., 757a, 757b) and a list of approved uses (e.g., 758a, 758b). For example, the quantity field can be used to determine in-stock, out-of-stock, amount on-order conditions as well as indicate a particular formulation. The particular formulation 752ao fields includes an identification or indication of a strain product type which can include but isn't limited to a concentrate, edible, tea, buds, oil, lotion, particular concentrations of active ingredients for a specific medical prescription/legal regulation, and the like. The list of approved uses 758 include applications such as “recommended for vaporizing,” “limited to topical application,” “for ingestion by mouth only,” and the like.

The vendor information manager 700 allows vendors (such as dispensaries) to quickly enter their inventory (“menu”). The vendor information manager 700 receives the name of a strain offered by the vendor and autofills and populates the associated fields for the vendor's menu using information for the selected strain retrieved from the strain knowledge base 500. The vendor can readily enter (or modify) vendor related information such as price. After the vendor completes entering the strain-related information, the strain is made (e.g., immediately) available for selection by the customer (e.g., as presented in a map by the strain vendor locator 800 discussed below) which helps to preserve the accuracy of time-sensitive data points (e.g., data relating to freshness, clinical testing, price) for access by prospective patients. The availability displayed by the strain finder 150 to one or more users can be contingent (e.g., filtered out depending) upon the vendor being in good standing with the site in regards to billing (e.g., where the vendor site pays license fees to the strain finder for the privilege of having the vendor products selectively located and presented by the strain finder site to customers highly likely to purchase the searched-for vendor product).

The vendor record 710 is optionally arranged to permit the vendor to provide scores (e.g., for symptoms and illnesses), where the strain knowledge base 500 can provisionally accept the ratings and/or modify the ratings with comparable data from previous ratings for the same or similar strains (e.g., from the same or similar users). The ratings can also be modified by a system administrator, for example, to replace and/or modify system or the (e.g., consensus averaged) strain knowledge base ratings for a particular strain/formulation being reviewed. In an embodiment, the effect of the optional vendor-supplied ratings is weighted by the vendor information manager is limited to the effect of a single (e.g., non-vendor) customer.

The vendor information manager 700 is arranged to provide “inventory management” functionality with verification provided by a feedback loop formed by customers providing indications concerning whether inventories presented to the customer (e.g., where the presented inventories are derived from information supplied by vendors) actually have the offered inventory item in stock. For example, (e.g., trusted, highly rated individual) customers can order product from a vendor and forward amounts purchased (and/or whether sufficient quantities exist to fulfill the order). When insufficient stock is present to fulfil an order, search results including the insufficiently stocked strain are removed so the strain is not visible (e.g., offered) to others (e.g., for a period of time or until the vendor makes effort to verify replenishment). The vendor information manager 700 also maintains a history of discrepancies so that vendors and/or vendor-associated users can be flagged and/or more closely monitored for potential fraud. For example, vendors and dispensaries can be penalized for listing non-existing inventory in response to a highly motivated user providing (e.g., electronic) feedback (e.g., via the customer information manager 600) to the strain vendor site that a particular vendor promoted an out-of-stock product for sale via the strain vendor site.

FIG. 8 is a logic diagram of a strain vendor locator 800 in accordance with embodiments of the present disclosure. The strain vendor locator 800 is a locator service such as provided by the strain vendor locator 430. The strain vendor locator 800 includes a strain vendor mapper 810 and a map (e.g., displayed and/or electronically instantiated) 850. The strain vendor locator 800 is arranged to present geo-spatial information to a customer, for example, searching for locations of vendors (e.g., suppliers, sellers, dispensaries, delivery services, and the like) with respect to one or more locations associated with (e.g., a present customer location) or specified by a customer.

The strain vendor locator 800 includes a map engine 812, vendor map 814, a tax mapper 816, a customer location 818, a search area 820, a jurisdiction mapper 822, and list of search results (e.g., 824a, 824b, 824c), where each search result 824 includes a vendor identifier (e.g., 824aa) and an associated cost (e.g., 824 ab) for a particular product (e.g., amount or “nugg” of a strain a term used for normalizing a price/quantity calculation for ease of comparison between different strains and respective amounts (“nugget”) of a strain (e.g.).

The map engine 812 is arranged to display, for example, a map 850, which is drawn to illustrate geographic features and political features (e.g., jurisdiction 852), a customer route or location 854, and identified vendors having a product identified with an icon (e.g., 856 and 868), where each such icon represents quantities such as the location of a vendor and relative cost of a product. The map engine 812 is also arranged to identify locations of vendors in a specific range or area related to the customer location.

The vendor map 814 is arranged to survey addresses (e.g., 722) of vendors and convert the address to geospatial coordinates for searching and display by the map engine. The tax mapper 816 is arranged to determine a tax amount by summing tax rates of overlapping jurisdictions (state, local, federal tax rates) for particular vendor (e.g., where the summed tax rate is included in a cost estimate presented to the customer).

The customer location 818 and the search area 820 (which can be accessed from the customer record 610) are used by the strain vendor mapper 810 to determine areas to search and to display as the map 850. The jurisdiction mapper 822 determines rules and regulations for a regulated search items and, for example, suppresses search results that might otherwise result in a violation for a customer (e.g., buying a quantity of product permitted for medical use but not recreational use when the customer has no prescription or medical marijuana card).

The strain vendor mapper 810 is arranged to optionally search for vendors based on operating hours (e.g., 730), to facilitate finding, for example, 24-hour facilities (which can be uniquely displayed by filtering or icon color). The strain vendor mapper 810 is arranged to optionally monitor the present location of the customer (e.g., via cell phone application and coordinates or IP address detection on the device being used) so, for example, the customer can be notified (e.g., via text message or email) that a searched-for strain is available nearby that matches a saved search with a high percentage. The strain vendor mapper is arranged to optionally calculate tradeoffs between distance to the customer and closeness of matching of search terms to a particular strain.

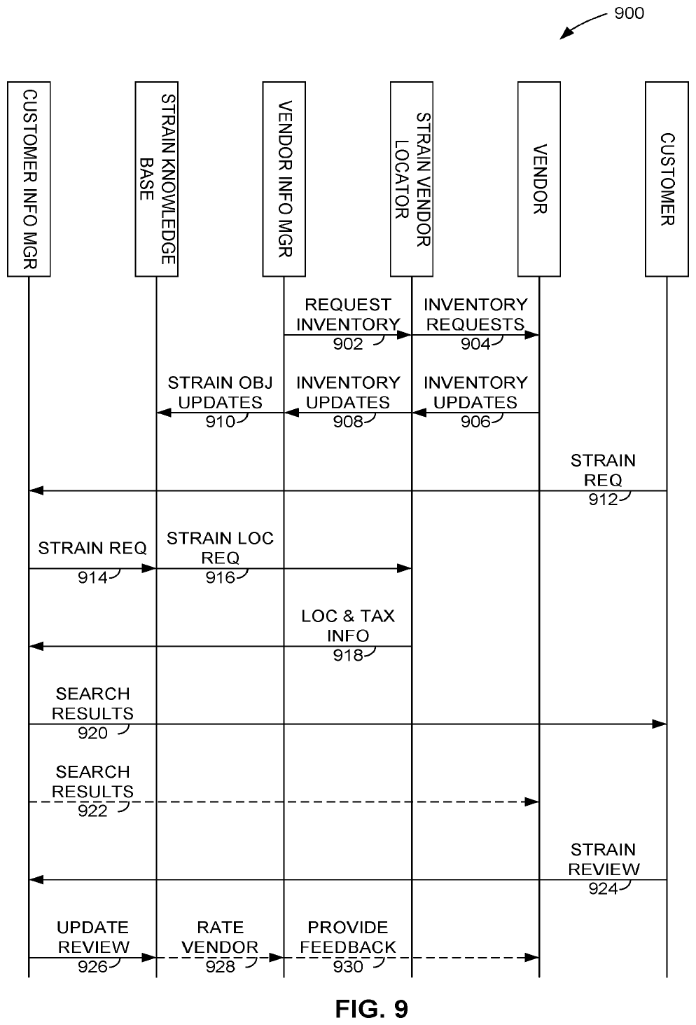

FIG. 9 is a signal flow diagram of a cultivar strain finder in accordance with embodiments of the present disclosure. Flow diagram 900 includes signal communications between users of a cultivar strain finder (e.g., a customer and a vendor) and components of the cultivar strain finder (e.g., strain vendor locator, vendor information manager, strain knowledge base, and customer information manager).

In communication 902, the vendor information manager sends an inventory request to the strain vendor locator. In communication 904, the strain object locator selects a vendor for inventorying and forwards inventory requests to the selected vendor. In communication 906, the vendor responds by sending an inventory updates to the strain vendor locator. The strain vendor locator updates records for determining a location having a particular strain in an inventory and, in communication 908, forwards the inventory updates to the vendor information manager. The vendor information manager updates a catalog (e.g., “menu”) for the selected vendor and, in communication 910, forwards strain object updates to the strain knowledge base for future searching.

In communication 912, the customer sends a search request for a strain in accordance with selected search parameters. The customer information manager records the search request (in association with the particular customer) and, in communication 914, sends a strain request with the selected the search parameters to the strain knowledge base. The strain knowledge base evaluates the receive search request and identifies particular strains closely matching the supplied search request parameters. In communication 916, the strain knowledge base forwards the request to the strain vendor locator.

The strain vendor locator determines a location of vendors, associated tax information and quantity restrictions responsive to particular user qualifications (e.g., such as age, prescription for active ingredients, medical marijuana card, and the like two). For example, a prescription can indicate that the product is not to be taxed, whereas lack of a prescription indicates an appropriate tax is to be charged. In communication 918, the strain finder locator forwards the determined information to the customer information manager for recording. In communication 920, the customer information manager sends the search results to the customer for example, in the form of a ranked list and/or map in which the location, relative cost, operating hours, user reviews, and the like are indicated to the customer.

In operation 922, selected search results are optionally sent from the customer information manager to the vendor (e.g., in response to a customer command or preference), which informs the vendor of the customers interest (if not an actual order for a product selected from the search). The customer information manager is arranged to store the order information in association with the particular customer records.

In operation 924, the customer provides a review of a particular strain (e.g., purchased from the selected vendor). The customer information manager records the supplied strain review in association with a particular customer so, for example, future search requests initiated by the particular customer use the customer-supplied scores and strain reviews to help select a strain more suited for achieving a desired outcome specified in a future search request.

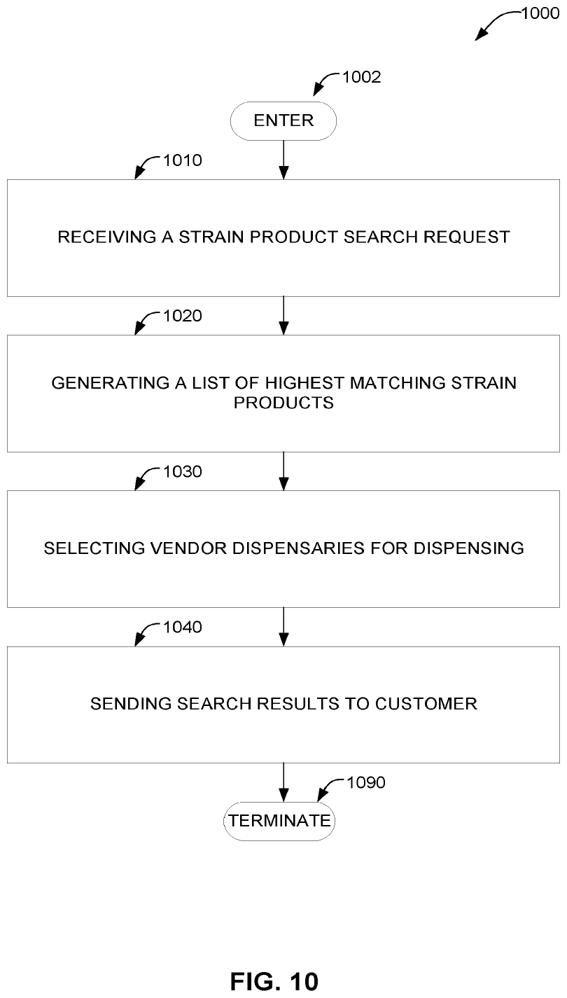

FIG. 10 is a flow diagram for finding strain products in accordance with exemplary embodiments of the disclosure. The program flow illustrated herein is exemplary, and thus various operations (and various portions of the operations) within the program flow can be performed concurrently and/or in an order that is not necessarily the same as the program flow illustrated herein (including, for example, using logical substitutions and reordering made in accordance with DeMorgan's theorems and Boolean algebra). Program flow 1000 begins at node 1002 and proceeds to operation 1010.

In operation 1010, a strain product search request is received from a particular customer, wherein the request is sent from a network-enabled device. For example, the particular customer can provide an electronic indication for indicating the particular customer intending to search for a strain product. Program flow proceeds to operation 1020.

In operation 1020, a processor is used to generate a list of highest matching strain products in response to searching a strain objects database for strain products. Each strain product is associated with a set of stored ranked outcomes. The highest matching strain products are determined by comparing each of the sets of stored ranked outcomes with a set of ranked outcomes determined for the customer and are determined by selecting the highest matching strain products associated with the highest matching stored ranked outcomes. Each set of ranked outcomes includes a plurality of diverse outcomes, with each such diverse outcome having a ranking.

For example, the set of ranked outcomes determined for the customer can be in included in the strain product search request, provided as a default in a record associated with the customer, a default provided for the customer, and the like. Program flow proceeds to operation 1030.

In operation 1030, vendor dispensaries for dispensing at least one of the highest matching strain products are selected in response to querying a vendor inventory, where the vendor inventory includes at least one associated vendor dispensary for dispensing at least one of the highest matching strain products. For example, a vendor can be associated with (e.g., own or control) many dispensaries, where each dispensary is typically a brick-and-mortar storefront (e.g., associated with a physical address) and/or delivery service (e.g., associated with a “location” such as a delivery area, route, franchise rights to a mapped area, and the like). The vendor inventory is associated with a vendor dispensary having in stock the highest matching strain products for dispensing to the customer. Program flow proceeds to operation 1040.

In operation 1040, the search results are sent to the customer, where the search results are determined in response to a location of each of the selected vendor dispensary and a customer location associated with the particular customer. For example, the customer location can be a residence, a delivery location, a route (e.g., commonly traveled by the customer), business address, and the like. Program flow proceeds to operation 1090.

In operation 1090, processing of the search request is terminated. In various embodiments, processors await further requests to be processed (often while multitasking other requests).

Details Regarding Terms Associated with Strain Recommendation Method Used by Strain Finder 150

strain profile—this reflects the desired characteristics that a user (e.g., customer 120, 130, 140) is seeking in a product. The user (e.g., customer 120, 130, 140) uses electronic devices to transmit inputs (e.g., via network 100) to strain finder 150 that uses the Strain Recommendation Method to identify and locate products including determining geographical locations of products that match these criteria found in the strain profile, or in other words, other strains that resemble the depicted strain profile.

strain recommendation—this is the result of the strain finder 150 using the Strain Recommendation Method, which is shown by strain finder 150 as a list of strains that resemble the desired characteristics that the user (e.g., customer 120, 130, 140) initially inputs (e.g., via network 110) to strain finder 150.

user_markers—these are the individual elements that comprise the strain profile. The user_markers (e.g., as network-based communicated to be received by such as strain finder 150) are related to the potential effects, medical benefits, side effects or the classification of a particular strain. Not only does the user (e.g., customer 120, 130, 140) transmit information (e.g., via network 110) to define the particular aspects of a desired profile (e.g.—sleepy), but the user (e.g., customer 120, 130, 140) also transmits inputs (e.g., via network 110) to strain finder 150 the desired magnitude of each characteristic (to continue using the example of ‘sleepy,’ a strain can be a little sleepy or very sleepy, with a spectrum of 1-5 (e.g., degrees ranging between 1 and 5).

classification_label—a subset of the defined user_markers, classification_label corresponds to the category that a particular strain belongs to: e.g., an indica, sativa, or a hybrid

desired_effects—a subset of the defined user_markers, desired_effects corresponds to one of the effects that could be selected by a user (e.g., customer 120, 130, 140) to input (e.g., via network 110) to strain finder 150 as corresponding to a strain: e.g., creative, energetic, euphoric, focused, good humored, happy, hungry, relaxed, sleepy, stimulated, talkative, tingly, uplifted, or others. This list will be modified as time goes by. Each desired_effect is input with a desired magnitude on a spectrum of 1-5 (1 being low and 5 being high).

medical_benefits—a subset of the defined user_markers, medical_benefits corresponds to one of the results that is identified as being produced by a particular strain. These include, but are not limited to e.g., help with depression, help with muscle spasms, help with insomnia, help with seizures, help lowering eye pressure, reduction or elimination of fatigue, reduction or elimination of headaches, help combatting insomnia, help with reducing inflammation, help with the reduction of nausea, help with the reduction of stress, help with alleviating cramps, help with reducing or eliminating pain, and help to restore appetite. As new therapeutic applications are identified, this list is going to be modified.

side_effects—a subset of the defined user_markers, side_effects are qualities that are considered negative which are identified as byproducts of certain strains. These are considered to be e.g., anxiety, dizziness, dry eyes, dry mouth, grogginess, headache, hunger, laziness, paranoia or confusion. This list is going to be modified over time as new side effects may emerge in the future.

reference_profile—this is the combination of the classification, desired effects, medical benefits, and/or side effects (e.g., as represented by reference_profile data vectors(s)) that a user (e.g., customer 120, 130, 140) is seeing. It is the totality of the input given (e.g., via network 110) by the user (e.g., customer 120, 130, 140) and represents a template that the method seeks to replicate from other strains it has knowledge of. The combination of classification, desired effects, medical benefits and side effects could mean any strain-related characterizing selections of up to 5 of the desired effects or medical benefits, in addition to the side effects as well as a restriction based on the classification.

strain_profile—a strain_profile is a strain that is identified and characterized (e.g., described with characteristic data) in strain knowledge base 434 of strain finder 150. A strain profile may include or may not include the desired effects associated with said strain (e.g., up to 13 of them), medical benefits associated with the strain (e.g., up to 13 of them), as well as any associated side effects (e.g., up to 10). The classification is always present for every strain profile.

strain knowledge base 434 of strain finder 150—this includes such as the collection of information for a number of strains that includes characteristics about their cannabinoid and terpene content, desired_effects, medical_benefits, side_effects, classification, and other information that could be relevant to a user (e.g., customer 120, 130, 140).

match_score—this is the calculated value that is derived when the method calculates the delta between the strain_profile a user inputs and a target strain in the strain knowledge base 434 (e.g., database) of strain finder 150 (e.g., database operating on at least one computer server). Expressed in %, it is calculated and can be viewed for every known strain in our database. A user (e.g., customer 120, 130, 140) must be logged (e.g., via network 110) in with an owned account in order to see the sort list.

delta—this is the difference between values of a strain. It is calculated by strain finder 150 based on a strain_profile a user inputs and this profile is compared to the profile of every known strain. Once the delta is identified by strain finder 150, it is converted (after the application of our weighted calculation to it) by strain finder 150 into a match score which is a measure of relative similarity between the reference_profile and any particular strain compared against it.

ad hoc matching calculation weighting_system—this is the collection of mathematical adjustments generated ad hoc by strain finder 150 from received (e.g., via network 110) based in part on strain-related characterizing selections including user_markers involved with subsequent calculation of standardized ranking system of strains relative to a particular set of parameters being sought out (e.g., via network 110) by a user (e.g., customer 120, 130, 140). The ad hoc calculation weighting_system generated ad hoc by strain finder 150, applies a sophisticated series of mathematical adjustments based on factors such as how a given strain differs from the sought out characteristics. In the process of analyzing the difference, different weights are assigned by strain finder 150 depending on the vector of the differences (does a strain offer more or less intensity then desired, the amount of difference in the intensity of an indicated characteristic (measured from 1-5 with the highest value indicating highest intensity), and applying a factor that correlates to the magnitude of a particular effect, therefore underscoring the value of high-impact strains. Additionally, different weights through generation of the ad hoc matching calculation weighting_system are assigned by strain finder 150 to effects, benefits, side effects (the adjustment for which is calculated as a negative correction), and the classification of the strain.

vector—this is used by strain finder 150 to identify how a characteristic within a reference_profile differs from a strain it is being compared to. If the chosen input (5) is higher than a strain's baseline (4), the vector is defined as a positive. If a user (e.g., customer 120, 130, 140) inputs (e.g., via network 110) to strain finder 150 a value (2) that is lower than the baseline (3), the vector is negative.

value—this is used by strain finder 150 to identify the actual intensity of a particular strain. A value of 5 is the characterization of maximum value, for the highest possible intensity of a particular effect, medical benefit, or a side effect. A value of zero is indicative of an absence of said attribute. For example, a strain with a value of 1 for the attribute of sleepy is very lightly sedating and is likely to make a user a little bit drowsy, but is unlikely to cause sleep. However, a strain with a sleepy value of 5 is very likely to cause a user to fall asleep.

magnitude—this calculation by strain finder 150 functions as a special adjustment for instances when values either approach their polar limits, or the delta between a characteristic of a reference_profile and an eligible match approaches the maximum possible difference in value. As magnitudes increases, an adjustment is applied as part of the calculation.

strain_recommendation—this is the final output by strain finder 150 of the Strain Recommendation Method, expressed as a list of strains, sorted by the calculated match score and listed in descending order (e.g., subsequently outputted strain_recommendation via network-based communication via network 110 based at least in part upon strain candidate scores).

Sample results can be outputted by strain finder 150 as follows:

Strain name—match score (highest)

Strain name—match score (medium)

Strain name—match score (lowest)

user_location—this is the geographic location (such as identified as associated with the Internet Protocol (IP) or other electronic address of a user's mobile or other electronic device) of a user (e.g., customer 120, 130, 140) at the time of using strain finder 150 with the Strain Recommendation Method. Through input to strain finder 150, a user (e.g., customer 120, 130, 140) can manually set (e.g., via network 110) the user's location identified to strain finder 150 by over-writing the auto-detected address.

proximity_setting—this is an input (e.g. via network 110) that user (e.g., customer 120, 130, 140) identicates to strain finder 150 of user's geographical radius (e.g., in miles, kilometers, meters, feet, etc.) from which strain finder 150, using Strain Recommendation Method, uses as a factor of strain eligibility in determining search results. Strains located outside the geographic radius set by the user will not be recommended by strain finder 150 using the Strain Recommendation Method.

availability—this is used by strain finder 150 to reference the action of looking up the locations (businesses) to which strains are mapped. This is based on inventory of strain products and is controlled at the retail or administrative level of strain finder 150. Strains that are not available for purchase (not in inventory) will not be outputted by stain finder 150 as among search results, even these unavailable strains have a very high match score relative to the reference profile.

Method 1: Strain Recommendation Method

Under this Strain Recommendation Method, the strain finder 150 parses a lot of complex factors and a wealth of information through purposefully architected strain knowledge base 434 in order to output a list of product rankings in a way that will help a user (e.g., customer 120, 130, 140) compare (e.g. via network 110) and select the most appropriate product(s) through input transmit (e.g. via network 110) to strain finder 150. Strain products vary greatly in their effects so the strain finder 150 helps users (e.g., customer 120, 130, 140) identify products (e.g. via network 110) that are most likely to provide desired effects. “Match Scores” are used by the strain finder 150 in order to communicate (e.g. via network 110) to user (e.g., customer 120, 130, 140) the likelihood that a particular product would fit the specific requirements of a particular user.

In order to utilize use of the Strain Recommendation Method by the strain finder 150 for finding products, a user (e.g., customer 120, 130, 140) must give, or input (e.g. via network 110) a set of parameters, to strain finder 150 so that the Strain Recommendation Method can output the appropriate product recommendations.

Following is an example of the Strain Recommendation Method as used by the strain finder 150:

#User Inputs Actively Inputted by User to Strain Finder 150 (Further Details):

1) Classification User Input

For each strain, a Classification Label (such as indica, sativa, or hybrid, which are in turn mutually exclusive and one applies to each strain, universally and independent of being subject of a user's search input (e.g. via network 110)) exists in knowledge base 434. Users (e.g., customer 120, 130, 140) can optionally include or ignore the Classification Label in their network-based communication (e.g. via network 110) to strain finder 150 in the consideration set of factors for calculating a “match score.” Strain finder 150 arranges products in a list based on descending order of match scores. This arrangement is what maximizes a product's relevance for an individual user (e.g., customer 120, 130, 140)—whether it coincides with desired product features/effects/outcomes. It is relevant to the aforementioned ‘parsing’ or outputting of information by the strain finder 150 relative to a user's particular query. The Classification system of the strain finder 150 takes into account the relevant variables, calculates expected benefits to the user (e.g., customer 120, 130, 140) based on the individual need, and each time, uniquely organizes the relative products relative to this “match score.”

When the Classification Label is included in the user selection parameters, the Strain Recommendation Method used by strain finder 150 assigns a factor of importance to this label, and therefore a portion of the final score is weighted accordingly. That is, if a user selects a preference for a given class of strains, match scores for strains in the knowledge base 434 of strain finder 150 eligible to be shown for that particular search will reflect a weighted adjustment given to strains that match the selected Classification Label, hence a factor of importance. The selection of a Classification Label when conducting a search by the strain finder 150 is optional and when selected, the class reflects a specific mathematical adjustment (+X % to match score) for all returned strain results. For example, 10 strains with identical properties will return the equivalent match score for a user search inputted (e.g. via network 110) to strain finder 150 relative to effects, however, if a user (e.g., customer 120, 130, 140) transmits (e.g. via network 110) search input to include the “Classification Label” among the search parameters, eligible strain scores calculated by strain finder 150 will reflect the values relative to the effect selections, with the strains subject to an incremental weighting calculation in circumstances where Classification Label is specified among the users' search parameters.

2) Desired Effects User Input

Each strain has a table of Desired Effects with associated values (happy=4, sleepy=5, etc.) found in knowledge base 434 of strain finder 150. The numeric range applicable to any specific Desired Effect attribute (from zero to 5) represents the expected magnitude of Desired Effect, with zero representing a total absence of particular Desired Effects from a given strain, to the expectation of very strong Desired Effects of a particular type in instances were said Desired Effects are identified with a magnitude of 5.

The Strain Recommendation Method is used by strain finder 150 to compare user (e.g., customer 120, 130, 140) inputs (e.g. via network 110) to the strain finder 150 (effectively a Table of Desired Effects based on the desired strain's profile). The table of Desired Effects for a strain is static/pre-existing, however, if a predetermined threshold of users submit to the strain finder 150 contrary values for a particular strain, then strain finder 150 can adjust the Desired Effect value(s) for the particular strain. Otherwise, the table of Desired Effects for strain is independent of a user input so that data in the knowledge base 434 of the strain finder 150 is maintained to be reliable for the user population. When the Strain Recommendation Method is used, the strain finder 150 compares two individual Desired Effects tables against a users' criteria, mathematically calculating the match score. The ranking of match scores is effectively the comparison (e.g., mapping comparison) being done by strain finder 150 and the results ranked in descending order.

The Weighting System involved with the Strain Recommendation Method takes the following into consideration in order for strain finder 150 to calculate a “score” for the Desired Effects component of each strain; this value is in turn applied towards the final match score:

Desired Effect presence (contributes to a higher score for the Desired Effects component of each strain) and Desired Effect absence (contributes to a lower score for the Desired Effects component of each strain).

An adjustment is applied by the strain finder 150 which correlates to the difference in the value between the user (e.g., customer 120, 130, 140) input (e.g. via network 110) value to strain finder 150 and the existing value found in the knowledge base 434 of strain finder 150 for each strain (i.e. the magnitude of a particular Desired Effect). The purpose is for strain finder 150 to underscore the value of high-impact strains and highlight the efficacy of products in categories where they excel. By this, strain finder 150, additionally, assists individuals (e.g., customer 120, 130, 140) seeking specific, high magnitude Desired Effects in finding products that are most likely to satisfy user (e.g., customer 120, 130, 140) desires.

Example: If a user, through use of strain finder 150, is searching for a strain with an X Desired Effect of “5”, with all other factors being equal, strain A, with a value of “4” for X Desired Effect would receive a match score nearly 30% higher than strain B, with a value of “3” for the same Desired Effect.

A user could choose to indicate to strain finder 150 to include or ignore Desired Effects in the Strain Recommendation Method calculation, with Desired Effects values being assessed only in case a user (e.g., customer 120, 130, 140) chooses to include Desired Effects in their search criteria inputted (e.g. via network 110) to strain finder 150.

3) Medical Benefits User Input

The Strain Recommendation Method used by strain finder 150 treats Medical Benefits in the same way as it does Desired Effects by taking the optional medical benefit selections inputted (e.g. via network 110) by a user (e.g., customer 120, 130, 140) to strain finder 150—such as ‘reducing pain,’ ‘helping with insomnia,’ or ‘relieving depression,’ among others—with the strain finder 150 using the Strain Recommendation Method comparing them against the known Medical Benefits of each eligible strain in the strain knowledge base 434 to yield match scores for each eligible strain (e.g., strain candidate scores). The Strain Recommendation Method used by the strain finder 150 uses a matching calculation weighting system for Medical Benefits selections that is different from the matching calculation weighting system for other groupings of selections such as Desired Effects selections or Side Effects selections. Once a match score is compiled for a particular grouping of selections, the match score is averaged by the strain finder 150 using the Strain Recommendation Method with the match scores previously calculated by the strain finder 150 from any other groupings of selections that have been inputted to the strain finder 150 (such as groupings of selections concerning Classification Label, Desired Effects, or Side effects) to calculate a compounded match score.

A user (e.g., customer 120, 130, 140) could choose to include or ignore inputting (e.g., via network 110) Medical Benefits selections to the strain finder 150 to subsequently be included in or excluded from building a matching calculation weighting system by strain finder 150 using the Strain Recommendation Method. Consequently, calculations are made through an ad hoc generated matching calculation weighting system by the strain finder 150 in response to a user (e.g., customer 120, 130, 140) inputting (e.g. via network 110) to the strain finder 150 choices to include grouping of Medical Benefits selections in the user's search criteria inputted to strain finder 150.

4) Side Effects User Input

As discussed, in the case of Desired Effects and Medical Benefits, when using the Strain Recommendation Method, the strain finder 150 functions by ad hoc generation of a matching calculation weighting system to thereby increase match score for strains by correlating values. In contrast, for Side Effects, the strain finder 150 when using the Strain Recommendation Method functions by ad hoc generation of a matching calculation weighting system including inverse calculations. Side Effects represent any effects deemed (e.g. via network 110) negative and/or unwanted to a particular user (e.g., customer 120, 130, 140). Therefore, the presence of any such Side Effect factors would diminish the desirability of a particular product for a particular user (e.g., customer 120, 130, 140). Therefore, there is an inverse relationship between the presence of unwanted characteristics of user (e.g., customer 120, 130, 140) selected (e.g., via network 110) Side Effects that strain finder 150 consequently diminishes the overall match score, as opposed to strain finder 150 increasing the overall match score as in response by strain finder 150 to user (e.g., customer 120, 130, 140) selections (e.g., via network 110) of Desired Effects or Medical Benefits. For instance, when a user (e.g., customer 120, 130, 140) identifies (e.g., via network 110) to strain finder 150 a Side Effects factor to be avoided (i.e. dizziness), if such a factor is associated with a particular strain, strain finder 150 using the Strain Recommendation Method through ad hoc generation of a matching calculation weighting system determines a negative adjustment (i.e. a reduction) in resulting match score for the particular strain involved.

As the strain finder 150, through ad hoc generation of a matching calculation weighting system, increases the weighting of an undesired Side Effects factor based on user (e.g., customer 120, 130, 140) input (e.g., via network 110) to strain finder 150, strain finder 150 using the Strain Recommendation Method generates an ad hoc matching calculation weighting system to implement adjustments at a predetermined rate(s) to best reflect the magnitude of unwanted Side Effects and to highlight the presence of a potentially negative experience with a given product.

#Inputs (Detected by Strain Finder 150):

5) Location of User Input

The strain finder 150 using the Strain Recommendation Method performs an ad hoc determination of user (e.g., customer 120, 130, 140) location through information received (e.g. via network 110), and takes proximity to user of location of candidate strain into account (e.g., strain candidate data) when generating an ad hoc matching calculation weighting system to evaluate existence of products for recommendations through match score calculation using the generated ad hoc weighting system. This determination of user (e.g., customer 120, 130, 140) geographical location is accomplished by strain finder 150 through accessing or otherwise utilizing (e.g. via network 110) geo-location features of the user's device and/or browser, leveraging the detected Internet Protocol (IP) (e.g., network protocol information) or other electronic address of user's device, which consequently provides a user's physical location (e.g., geographical location) or a close approximation thereof (e.g., electronic device geographical location).

Users (e.g., customer 120, 130, 140) can modify personal settings inputted (e.g. via network 110) to strain finder 150 through the Strain Recommendation Method, which includes the option to modify (e.g. via network 110) the geographical radius of interest. Regardless of match, through generation of ad hoc matching calculation weighting systems by strain finder 150, a particular strain product will not be shown by strain finder 150 using the Strain Recommendation Method if the particular strain product is outside (e.g., lacking acceptability of geographical location based on geographical location data stored in such as strain knowledge base 434) of the geographic area of interest selected (e.g. via network 110) by user (e.g., customer 120, 130, 140).

6) Cannabinoid & Terpene Content Input

The strain finder 150 using the Strain Recommendation Method generates ad hoc matching calculation weighting systems to evaluate available strain products from the perspective of cannabinoid and terpene content relative to the user (e.g., customer 120, 130, 140) criteria inputted (via network 110) to strain finder 150 through the Strain Recommendation Method by mapping (e.g. mapping comparison) cannabinoid and terpene content data for particular candidate strain products (e.g., strain candidate data, such as data of candidate strain(s)) stored in strain knowledge base 434 of strain finder 150 to known Desired Effects and Medical Benefits data also stored in strain knowledge base 434 of strain finder 150 correlated with distinct potencies/concentrations/formulations naturally found in the grown and mature cannabis plant. Data for each strain may contain values for all known cannabinoids found in the strain (therefore, a table of values could be said to exist for every strain data object). When data is to be included for a new strain into the strain knowledge base 434 of strain finder 150, the cannabinoid content data of the new strain can be based on laboratory tests using Gas and Liquid Chromatography so that the data detailing the contents of each strain relative to known cannabinoids and terpenes is included with each strain data object based on test results. Based on the presence of cannabinoids in ratios recognized to be therapeutic relative to the user search criteria, the strain finder 150 using the Strain Recommendation Method applies a score adjustment.

As a representative example, when a user (e.g., customer 120, 130, 140) inputs (e.g. via network 110) selection criterion to the strain finder 150 that indicates to the strain finder 150 that the user (e.g., customer 120, 130, 140) seeks a strain product to aid with depression, the strain finder 150 using the Strain Recommendation Method could apply a 1.6× multiplier to the match score for strains with THC between 14%-18% and CBD 3%-4%. These ranges selected by the strain finder 150 through generation of ad hoc matching calculation weight system were used in this example due to data stored in strain knowledge base 434 regarding a large body of research and testing that indicate application of said compounds, in identified doses, has shown to be effective at achieving relief from the symptoms reported by depression sufferers.

Method 1: Example of Strain Recommendation Method Used by Strain Finder 150

1: #Input: list of criteria—reference_profile created

2: #Output: list of products—strain_recommendation

3: #Step 1: Analyze user_markers:

4: Get classification_label

5: Get desired_effects

6: Get medical_benefits

7: Get side_effects

8: Compile reference_profile

Note: Step 8 is performed by getting class, effects, benefits, etc. to compile the reference profile through generation of ad hoc matching calculation weight system

9: #Step 2: Compare reference_profile against each strain_profile in the database to calculate a match_score

10: for each strain_profile in the strain knowledge base 434 database do

11: Map reference_profile to cannabinoid_profile and apply adjustments as needed through generation of ad hoc matching calculation weighting system

That is, when the reference profile indicates through generated ad hoc matching calculation weight system a desired state, such as relief for depression, an eligible strain's cannabinoid_profile will be considered by strain finder 150 and assessed relative to contents, consequently determining if the contents positively or negatively affect the match score of that particular strain, relative to a reference_profile.

12: Compare reference_profile with strain_profile

When a reference profile indicates a desired state, such as relief for depression, an eligible strain's profile will be indexed by strain finder 150 for all attributes of interest as indicated in the reference_profile (in the present example, this would specifically refer to the «depression» attribute). Based on how the attributes compare, a match score is determined by strain finder 150 through generation of an ad hoc matching calculation weighting system, which, like the cannabinoid contents score, will be jointly considered in the ultimate measure of strain's compatibility to the users' preferences.

13: Calculate delta between strain_profile and reference_profile

The delta is calculated by strain finder 150 by measuring the difference between the presence of desired attributes among eligible candidate strain profiles (e.g., candidate strain_profile data vectors) in a user's given geographical radius, compared to a user's ideal attributes, as indicated by a reference profile.

The delta is not determined by strain finder 150 in absolute value, as the over-abundance of a particular effect is considered to create less disparity with a user's desired outcome, and therefore impose a lesser penalty by strain finder 150 to the match score than would be imposed by the absence of a desired effect. This is referred to below as a vector.

Furthermore, in calculating the delta by strain finder 150, generation of an ad hoc matching weighting system is applied by strain finder 150 to determining a match score as the range between a desired attribute from a reference profile to same attribute within a strain profile increases. The impact to the match score can consequently exceed linear growth.

For example: A candidate strain that differs from a reference profile by 2 value points is not penalized by strain finder 150 by a doubling of the penalty should that difference had been of only 1 value point.

14: for each delta calculation do

15: Apply ad hoc generated weighting_system and calculate match_score

16: for each delta, consider

17: Vector

18: Value

19: Magnitude

20: Compile match_score list in order of highest to lowest

Once strain finder 150 applies a generated ad hoc weighting system to calculate match scores for all eligible strain profiles, strain finder 150 outputs a list, ordered by the highest match score to the lowest.

21: #Step 3: Determine eligibility set for strain_recommendation

22: Get user_location

23: Identify user_location

24: Apply proximity_setting

A user input to strain finder 150 a geographical border that strain finder 150 uses to restrict the returned results included in generating an ad hoc matching calculation weighting system based upon this setting. That is, products outside of the geographical radius set by the user in the proximity setting inputted to strain finder 150 through the Strain Recommendation Method are not eligible for consideration, being too far, and are therefore excluded from search results.

25: Retrieve availability of strains based on product location and proximity setting through application of generated ad hoc matching calculation weighting system

26: Exclude any strain_profile ineligible for recommendation

strains that are outside the desired distance will be excluded

27: Step 4: Sort the strain_recommendation

The results are shown by strain finder 150 to user in order from highest match score to lowest match score.

28: Display strain_profile in descending order based on calculated match_score

Further Strain and Reference Profiles Details

Strain Variety (also known as strain classification) used by strain finder 150 can be defined as follows:

$$variety = \{ \begin{matrix}1 , & if strain is sativa \\ 2 , & if strain is indica \\ 3 , & if strain is hybrid\end{matrix}$$

Strain Effects vector used by strain finder 150 can be represented as follows:

$$E = ( \begin{matrix}{e}_{1} \\ {e}_{2} \\ {e}_{3} \\ ⋮ \\ {e}_{n}\end{matrix} )$$

$$0 <= {e}_{i} <= 5$$

Where n represents the number of the effects in the database and eᵢ represents a value or magnitude of the effect i specific to a specific Strain or Reference Profile.

Strain Medical Benefits vector used by strain finder 150 can be represented as follows:

$$MB = ( \begin{matrix}{mb}_{1} \\ {mb}_{2} \\ {mb}_{3} \\ ⋮ \\ {mb}_{m}\end{matrix} )$$

$$0 <= {mb}_{i} <= 5$$

Where m represents the number of medical benefits in strain knowledge base 434 of strain finder 150 and mbᵢ represents a value or magnitude of the medical benefit i specific to a specific Strain or Reference Profile.

Strain Negative Effects vector used by strain finder 150 can be represented as follows:

$$NE = ( \begin{matrix}{ne}_{1} \\ {ne}_{2} \\ {ne}_{3} \\ ⋮ \\ {ne}_{q}\end{matrix} )$$

$$0 <= {ne}_{i} <= 5$$

Where q represents the number of negative effects in strain knowledge base 434 of strain finder 150 and eᵢ represents a value or (sometimes “magnitude” in the doc) of the negative effect i specific to a specific Strain or Reference Profile.

Strain and Reference Profiles

Those three vectors and variety variable from the above sections represent the Strain Profile that can be electronically stored for every strain individually in strain knowledge base 434 of strain finder 150.

The strain knowledge base 434 of strain finder 150 can be populated through input from strain cultivators and values or magnitudes can be stored in strain knowledge base 434 of strain finder 150 for strain effects, medical benefits and negative effects already standardized. The scale can be chosen for every vector individually and/or particular values can be used such as on a scale from 0 to 5 for all strain effects, medical benefits and negative effects such as follows:

SPᵢ={v,E,MB,NE}

Where SPᵢ is a Strain Profile for Strain i.

Reference Profile associated with Strain Wizard as used by strain finder 150 (strainrx.co/search/strain/wizard/) has a similar definition to Strain Profile such as follows:

$$RP = \{ V , E , MB , NE \}$$

$$V = \{ {v}_{i} \}$$

$${v}_{i} = \{ \begin{matrix}1 , & if sativa is selected \\ 2 , & if indica is selected \\ 3 , & if hybrid is selected\end{matrix}$$

Strain Score Calculation Based on Reference Profile

This section clarifies how strains are scored by strain finder 150 based on the Reference Profile that is composed from the user's preferences input. As per the definitions above, there can be two similar sets of vectors, except for strain variety (strain classification) part that is used for filtering. What follows is an application of strain variety filter thereby making sets homogeneous.

Step 1: strain finder 150 applies Strain Variety filter, i.e. filter strains that match selected strain varieties.

As a result of step one, one attribute from the Reference/Strain Profile can be reduced by strain finder 150 with the assumption that the strains of the requested variety have already been filtered.

Consequently, two homogeneous sets of vectors remain that will be used for scoring:

RP={rE,rMB,rNE}

SPᵢ={Eᵢ,MBᵢ,NEᵢ}

Formula(s) for scoring by strain finder 150 (clarifies the effects, benefits and negative effects scores adjustments) are as follows:

$${score}_{i} = \frac{\begin{matrix}adjusted ( rE , {E}_{i} ) + adjusted ( rMB , {MB}_{i} ) + \\ negativelyAdjusted ( rNE , {NE}_{i} )\end{matrix}}{maximumScore}$$

$$maximumScore = {\sum }_{i = 1}^{n} {re}_{i} + {\sum }_{i = 1}^{m} {rmb}_{i}$$

Where reᵢ is the value of the effect i of reference profile and rmbᵢ is the value of the medical benefit i of reference profile.

The following formulas represent one or more weighting systems used by strain finder 150 that can be derived from statistical research involving strain knowledge base 434 to adjust score of strains depending on user search parameters such as follows:

$$adjusted ( rE , sE ) = {\sum }_{i = 1}^{n} ( {re}_{i} + bonus ( {re}_{i} , {se}_{i} ) + correction ( {re}_{i} , {se}_{i} ) )$$

$$bonus ( re , se ) = \{ \begin{matrix}0 , & re \neq se \\ 0.2 , & re = 3 \\ 035 , & re = 4 \\ 0.55 , & re = 5\end{matrix} \\ correction ( re , se ) = \{ \begin{matrix}0 , & re = se \\ - 0.01 , & re < se \\ re * fine ( re , se ) , & re > se\end{matrix} \\ fine ( re , se ) = \{ \begin{matrix}- 0.14 , & - 1 <= se - re < 0 \\ - 0.33 , & - 2 <= se - re < - 1 \\ - 0.51 , & - 3 <= se - re < - 2 \\ - 0.8 , & - 4 <= se - re < - 3 \\ - 1 , & - 5 <= se - re < - 4\end{matrix} \\ negativelyAdjusted ( re , se ) = \{ \begin{matrix}re = 0 or se = 0 \\ - 1 * \frac{{( re - se )}^{2}}{maximumScore}\end{matrix}$$

Further Steps of Strains Ranking

Step 2: strain finder 150 calculates scoreᵢ from above formula for filtered strains and order descendingly.

Step 3 (optional): if the user has selected the location filtering option then strain finder 150 checks each strain to determine if each strain is available in dispensaries near the user location. Strain finder 150 approximates user location by IP address or geolocation coordinates provided to strain finder 150 by user's device through consent by user.

Step 4 (for user-interface purposes only, so users don't see theoretical scores below 0 and above 100): strain finder 150 outputs list of strain suggestions from Step 2 and prettifyScore using the function below:

$$prettifyScore ( score ) = \{ \begin{matrix}0 , score < 0 \\ 100 , score > 100 \\ score\end{matrix}$$

Clarifications for Method 2: Cannabinoid/Terpene Analysis for Strain Recommendation Personalization and Cannabinoid & Terpene Content Analysis from Method 1: Strain Recommendation Method

Of note, “Method 2” generally is intended to be used by strain finder 150 as a complement to the weighting system from “Method 1”. Thus, the strain finder uses “Method 2” as a “cannabinoid & terpene content score adjusting (adjuster)” with an objective of strain finder 150 to incorporate the body of theoretical knowledge about cannabinoids, terpenes and their effects involved with “Method 2” into generation of ad hoc matching calculation weight system for the scoring system of strain finder 150 involved with “Method 1.”

### Definitions

As used by the strain finder 150, an extension of defined Strain Profile with Cannabinoids and Terpenes vectors is as follows:

$$C = ( \begin{matrix}{c}_{1} \\ {c}_{2} \\ {c}_{3} \\ ⋮ \\ {c}_{p}\end{matrix} )$$

Where p is the number of known cannabinoids defined in system used by the strain finder 150 and 0<=cᵢ<=1 is the decimal value of cannabinoid c content in a strain and Cᵢ is a vector of cannabinoids contents of strain i. The terpene vector as used by strain finder 150 can be defined as follows:

$$T = ( \begin{matrix}{t}_{1} \\ {t}_{2} \\ {t}_{3} \\ ⋮ \\ {t}_{r}\end{matrix} )$$

$${t}_{i} ϵ \{ 0 , 1 \}$$

Where r is the number of known terpenes defined in system used by the strain finder 150 and tᵢ is the binary value that strain finder 150 uses to determine if terpene i is present in the strain and Tᵢ is a vector used by strain finder 150 for terpene contents of strain i.

An adjustment procedure including an ad hoc generation of matching calculation weighting system used by strain finder 150 to adjust effectScore is as follows: if elementRequirements are satisfied by strainContent then multiply effectScore by multiplier. Where elementRequirements is a list of ranges for cannabinoid contents and terpenes.

Example of how strain finder 150 implements this adjustment procedure:

LL1%<=Cannabinoid X Content %<=RR1%

LL2%<=Cannabinoid Y Content %<=RR2%

Terpene B is present (t[i] takes value of 1) (since Terpenes vector is defined by binary values). This adjustment procedure can affect only effectScore for a specific effect.

Adjustment Procedure Definitions

The following definitions the adjusted and negativelyAdjusted functions are used by the strain finder 150 to work with the adjustment rules:

$$adjusted ( rE , sE , sC , sT ) = {\sum }_{i = 1}^{n} contentAdjustment ( {re}_{i} + bonus ( {re}_{i} , {se}_{i} ) + correction ( {re}_{i} , {se}_{i} ) , sC , sT )$$

$$negativelyAdjusted ( re , se , sC , sT ) = \{ \begin{matrix}0 , re = 0 or se = 0 \\ - 1 * contentAdjustment ( \frac{{( re - se )}^{2}}{maximumScore} , sC , sT )\end{matrix}$$

Where sC is cannabinoid contents of a strain and sT is terpene content of a strain. Also, contentAdjustment(effectScore, sC, sT) is the method used by the strain finder 150 that finds the rule with the biggest effect multiplier to the specific effect (or medical benefit or negative effect) that corresponds to strain cannabinoid and terpenes content and applies that multiplier.

contentAdjustment Pseudocode (Describing Operations of the Strain Finder 150):

effectRules=allRules|filtered, specific to the effect e[i]

maxMultiplier=1

strainContents={sC, sT} #set of Cannabinoid and Terpene vectors

for rule in effectRules:
contentMatch=true
for contentRequirement in rule.contentRequirements:
   if not contentRequrement.satisfied(strainContents):
      contentMatch=false
if contentMatch:
   maxMultiplier=max(maxMultiplier, rule.multiplier)

effectS core=effectScore*maxMultiplier

contentRequrement.satisfied(strainContents) pseudocode:

Object contentRequirement:
method satisfied(strainContents):
   for elementRequirement in this.requirements:
      If terpene from elementRequirements is not in strainContents OR cannabinoid from strainContents is not in range of elementRequirements:
         return false
   return true

Method 2: Cannabinoid/Terpene Analysis Method for Strain Recommendation Personalization

The strain finder 150 uses a Cannabinoid/Terpene Analysis Method for generation of ad hoc matching calculation weighting system to evaluate cannabinoid and terpene content of a strain_profile to personalize strain recommendations based on a user's (e.g., customer 120, 130, 140) desired criteria inputted (e.g. via network 110) to strain finder 150. Cannabinoid/Terpene Analysis Method aids users in finding the optimal products that best suit their individual needs. The amount and ratio of cannabinoids and terpenes present in a strain is a major indication of a strain's therapeutic potential. Therefore cannabinoid content should play a role in a strain recommendation.

When a user (e.g., customer 120, 130, 140) inputs (e.g. via network 110) a search criteria (ie creates a reference_profile) to strain finder 150—which may include one or more Desired Effects, Medical Benefits, or Side Effects (collectively, characteristics)—strain finder 150 uses the Cannabinoid/Terpene Analysis Method to generate an ad hoc matching calculation weighting system to incorporates analysis of eligible strains' cannabinoid_content and consequently adjusts match_score values to complement the reference_profile. Through use of Cannabinoid/Terpene Analysis Method, Strain finder 150 generates an ad hoc matching calculation weighting system by referencing a mapping (e.g., mapping comparison) database of strain knowledge base 434 of strain finder 150 that contains rules which govern the relationships between cannabinoid_profiles and characteristics. Strain finder 150, using Cannabinoid/Terpene Analysis Method, then follows those rules to generate ad hoc matching calculation weighting system to apply adjustments to base_values of eligible strain_profiles before a final match_score is compiled by strain finder 150.

Specific cannabinoid_profiles are mapped by strain finder 150 to individual characteristics in strain knowledge base 434 of strain finder 150 which strain finder 150 uses to describe how a cannabinoid_profile interacts with characteristics via a series of rules. Accordingly, strain finder 150 can interact various cannabinoid_profiles with various characteristics in enhancing or a lessening ways through generation of ad hoc matching calculation weighting system. The creation of a reference_profile by strain finder 150 triggers, through the Cannabinoid/Terpene Analysis Method, strain finder 150 to search strain knowledge base 434 for existing rules governing relationships between cannabinoid_profiles and the characteristics selected in the reference_profile, to subsequently assemble results that relate to cannabinoid content in addition to all other factors in making a ranked recommendation.

The rules that strain finder 150 uses to govern the dynamic relationships between cannabinoid_profiles and characteristics are based on data contained in strain knowledge base 434 associated with a growing body of knowledge and understanding about how cannabinoids and terpenes interact with human physiology so that changes and additions with future advances can be inputted into strain knowledge base 434. An example of one such rule used by strain finder 150 is that when a cannabinoid_profile contains a CBD concentration over 7%, through the Cannabinoid/Terpene Analysis Method, strain finder 150 raises the base_value of the “alleviate pain” characteristic by 13% in the event that the reference_profile includes the “alleviate pain” characteristic. In this example, this rule found in strain knowledge base 434 is supported by published medical findings stating that a sufficient concentration of CBD is known to have analgesic effects in cannabis strains. Additional rules describing how cannabinoid_profiles impact characteristics are contemplated to be included in strain knowledge base 434 based on advances in the medical literature.

Examples of additional rules describing how cannabinoid_profiles impact characteristics are as follows:

### Example 1

When reference profile includes “creative,” apply a 1.5× multiplier to the base_value of “creative” if CBD is >3% and a THC:CBD ratio is between 3:1 and 4:1.

### Example 2

When reference profile includes “alieve pain,” apply a 1.2× multiplier to the base_value of “alieve pain” if THC>15%, or apply a 1.2× multiplier to the base_value of “alieve pain” if THC>25%. Adjustments are not cumulative; apply only the calculation with the greatest positive impact on resulting base_value.

### Example 3

When reference profile excludes “hungry” (side effect), apply a −1.3× multiplier if THC>14%

If a user wishes to avoid hunger inducing products, a strain with THC of over 14% that did not have “hungry” identified gains a value of 1, while a strain that had a value already identified for “hungry” has that existing value multiplied by 1.3. Results of calculations are always rounded to the nearest whole number.

Cannabinoid/Terpene Analysis Method Example

1: #Step 1: Analyze reference_profile

2: Detect desired characteristics [e: “creative”, m: “pain_relief”, s: “hungry”]

3: #Step 2: Rule Retrieval

4: for each strain_profile in the strainrx database do

5: Scan cannabinoid_profile

6: Retrieve rules correlating to the cannabinoid_profile

7: Identify rules related to characteristics in reference_profile and exclude others

8: #Step 3: Execute Adjustment through generation of ad hoc matching calculation weighting system

9: Execute selected rule(s) and adjust correlated base_value(s)

Characteristics—also referred to as user_markers, these constitute the various effects, benefit, or side effects that go with various strain_profiles

Cannabinoid_profile—a cannabinoid profile is the molecular contents of a strain. It is unique and exact, like a fingerprint.

Base_values—these refer to the value assigned to a characteristic of a strain profile before any adjustments are made to it.

Details of Terms Involved with Method 3: User Feedback Handling Method for Self-Correction & Learning

characteristics—the most basic concept associated with the ideas of effects, medical benefits, and side effects is this concept of characteristics. The basic elements that make up effects include ‘happy,’ ‘energetic,’ and the like. Medical Benefits and Side Effects are also comprised of many such characteristics. This example list is envisioned to expand over time to further capture the gamut of human experiences. There are defined for all strain_profiles and enable users (e.g., customer 120, 130, 140) to include every defined characteristic for search purposes.

strain_profile—a strain_profile is a strain that is identified and characterized with attributes as exists in our database.

n—this refers to a sample size, or a total number of inputs considered for applying an adjustment to an existing value within the strain knowledge base 434 of strain finder 150.

novel_criteria—this refers to a value this is received in the form of user (e.g., customer 120, 130, 140) feedback (e.g., via network 110) which we are enabling users (e.g., customer 120, 130, 140) to provide for every strain_profile that differs from the value strain finder 150 identifies.

new_value—this is referring to the calculated value (outcome) of a calculation in a particular method which is then applied to a previously defined base_value for a given characteristic in a strain_profile.

base_value—this refers to the value identified and published by strain finder 150 for a specific characteristic within a specific strain_profile.

vector—this refers to the directionality of value(s) relative to base_value(s). If a change is submitted that reflects a value that is higher than a base value, the change is said to have a positive vector. If a change is submitted that reflects a value that is lower than a base value, the change is said to have a negative vector.

delta—this is the difference between values of a strain. It is calculated based on a strain_profile a user (e.g., customer 120, 130, 140) inputs (e.g. via network 110) and this profile is compared by strain finder 150 to the profile of every known strain. Once the delta is identified, it is converted (after the application of our weighted calculation to it) into a match score which is a measure of relative similarity between the reference_profile and any particular strain compared against it.

Method 3: User Feedback Handling Method for Self-Correction & Learning

If any Desired Effect, Medical Benefit, or a Side Effect (collectively, “characteristics”) is not initially recorded by strain finder 150 for a strain_profile but is later contributed by n users to strain finder 150 via the “edit” functionality, strain finder 150 learns from this feedback and automatically updates the target strain_profile with the novel characteristic(s) to reflect the users' (e.g., customer 120, 130, 140) inputs (e.g. via network 110). After defining n based on a determination of statistical significance, the average of n inputs (e.g., via network 110) is calculated through use of the User Feedback Handling Method by strain finder 150. Strain finder 150 then adds the characteristic to the strain_profile with a value equal to the calculated average of the n inputs, rounded to the nearest whole number.

User Feedback Handling Method Example

1. #Retrieve: existing strain_profile

2: Define n

3: if

4: #Input novel_criteria for existing strain_profile n times

5: then

6: Calculate average novel_criteria for a characteristic across n inputs:

7: (novel_criteria1+novel_criteria2 . . . +novel_criterian)/n=new_value

8: Apply new_value for the characteristic to the strain_profile

For characteristics already defined by strain finder 150 within particular strain_profiles, in case of receipt of novel_criteria via the “edit” feature at the hands of the users (e.g., customer 120, 130, 140), strain finder 150 updates the characteristic value so as to reflect user feedback (e.g., via network) in real time, thereby learning as more data is collected.

First, through use of User Feedback Handling Method, strain finder 150 collects submitted novel_criteria in blocks of n, where n is determined to be statistically significant. All feedback (as sequentially received) is grouped by strain finder 150 by vector (positive or negative direction relative to the base_value of a characteristic in a strain_profile). When sufficient inputs of a uniform vector are received through User Feedback Handling Method, the average of these inputs is calculated by strain finder 150. Then strain finder 150 uses the User Feedback Handling Method to determine the % difference between the calculated average of the received inputs and the base_value of a given characteristic in a target strain_profile. Once this is calculated by strain finder 150 through User Feedback Handling Method, strain finders 150 uses User Feedback Handling Method to adjust the base_value by the calculated % delta (in the respective vector). Once the % adjustment is applied, the calculation is rounded by strain finder 150 to the nearest whole number and the new value is output as the new measure of the said characteristic. Through User Feedback Handling Method, stain finder 150 collects feedback in each vector into blocks of n inputs and each time n number of inputs in a given vector is collected, an adjustment by strain finder 150 is carried out.

User Feedback Handling Method Example

1. Define n

2. if

3: #Input novel_criteria for existing characteristic in a strain_profile n times

4. then

5. Group n sequential inputs of novel_criteria of a uniform vector

6. Calculate average novel_criteria for a characteristic across n inputs

7. (novel_criteria1+novel_criteria2 . . . +novel_criterian)/n=new_value

8. Calculate % delta between new_value and base_value

9. Apply % adjustment to base_value in the appropriate vector

Method 4: Method to Translate Match Scores to the Kiosk

For the Kiosk, the % matching system is simplified and expressed with one of 5 labels. These labels correlate to the match score.

“Great Match”=100-90%

“Good Match”=89-80%

“Fair Match”=79-68%

“Poor Match”=67-50%

“Bad Match”=<50%

Method to Translate Match Scores to the Kiosk Example

1. If

2. Match score=[value range 90+]

3. Then

4. Print “Great match”

5. If

6. Match score=[value range 89-80]

7. Then

8. Print “Good Match”

9. If

10. Match score=[value range 79-68]

11. Then

12. Print “Fair match”

13. If

14. Match score=[value range 67-50]

15. Then

16. Print “Poor Match”

18. If

19. Match score=[value range <50]

20. Then

21. Print “Bad match”

The Method to Translate Match Scores to The Kiosk can be used by strain finder 150 for tablet installation at the point of retail. To aid customers using strain finder 150 in making decisions easier, the match score outputted by strain finder 150 uses labels corresponding to numeric values internally calculated by strain finder 150.

The various exemplary embodiments described above are provided by way of illustration only and should not be construed to limit the claims attached hereto. Those skilled in the art will readily recognize various modifications and changes that could be made without following the example exemplary embodiments and applications illustrated and described herein, and without departing from the true spirit and scope of the following claims.

What is claimed is:

1. A machine comprising:
at least one computer-based server;
at least one database operating on the at least one computer-based server;
at least one processor;
at least one non-transitory memory storage device storing at least one non-transitory computer instruction configured to instruct the at least one processor to perform including
receiving at least one network-based communication regarding strain-related characterizing selections;
generating at least one ad hoc matching calculation weighting system based at least in part upon the receiving at least one network-based communication regarding strain-related characterizing selections;
using the at least one ad hoc matching calculation weighting system with strain candidate data stored in the at least one database to at least in part determine at least one strain candidate score for at least one strain candidate; and
outputting at least one network-based communication regarding at least one strain recommendation based at least in part on the at least one strain candidate score.

2. The machine of claim 1, wherein the at least one non-transitory memory storage device storing at least one non-transitory computer instruction configured to instruct the at least one processor to perform including the receiving at least one network-based communication regarding strain-related characterizing selections including at least one of the following: at least one classification label, at least one desired effect, at least one side effect, and at least one medical benefit.

3. The machine of claim 1, wherein the at least one non-transitory memory storage device storing at least one non-transitory computer instruction configured to instruct the at least one processor to perform including
the generating at least one ad hoc matching calculation weighting system based at least in part upon the receiving at least one network-based communication regarding strain-related characterizing selections further including
compiling a reference_profile data vector from the receiving at least one network-based communication regarding strain-related characterizing selections; and
performing a mapping comparison between the reference_profile data vector and each of a plurality of candidate strain_profile data vectors to determine vector differences for each of the plurality of candidate strain_profile data vectors.

4. The machine of claim 1, wherein the at least one non-transitory memory storage device storing at least one non-transitory computer instruction configured to instruct the at least one processor to perform including
receiving at least one network-based communication regarding network protocol information related to an electronic device geographical location;
determining the electronic device geographical location based on the network protocol information;
comparing the electronic device geographical location to a geographical location associated with at least one candidate strain according to geographical location data stored in the at least one database to determine acceptability of the geographical location associated with the at least one candidate strain; and
outputting at least one network-based communication regarding at least one strain recommendation based at least in part on the acceptability of the geographical location associated with the at least one candidate strain.

5. The machine of claim 1, wherein the at least one non-transitory memory storage device storing at least one non-transitory computer instruction configured to instruct the at least one processor to perform including receiving at least one network-based communication regarding strain-related characterizing selections includes receiving user_markers associated at least in part with at least one magnitude of strain-related characterizing selections.

6. The machine of claim 1, wherein the at least one non-transitory memory storage device storing at least one non-transitory computer instruction configured to instruct the at least one processor to perform including
generating the at least one ad hoc matching calculation weighting system based at least in part upon at least one rule that governs at least one relationship between cannabinoid_profiles and at least one characteristic, the at least one characteristic selected from one or more of at least one Desired Effect, at least one Side Effect, and at least one Medical Benefit.

7. The machine of claim 1, wherein the at least one non-transitory memory storage device storing at least one non-transitory computer instruction configured to instruct the at least one processor to perform including
modifying characteristic data of at least one strain_profile based at least in part upon the receiving at least one network-based communication regarding strain-related characterizing selections.

8. A method being implemented via execution of non-transitory computing instructions configured to run on one or more processors and stored on one or more non-transitory computer-readable media, the method comprising:
receiving at least one network-based communication regarding strain-related characterizing selections;
generating at least one ad hoc matching calculation weighting system based at least in part upon the receiving at least one network-based communication regarding strain-related characterizing selections;
using the at least one ad hoc matching calculation weighting system with strain candidate data stored in the at least one database to at least in part determine at least one strain candidate score for at least one strain candidate; and
outputting at least one network-based communication regarding at least one strain recommendation based at least in part on the at least one strain candidate score.

9. The method of claim 8, wherein the receiving at least one network-based communication regarding strain-related characterizing selections includes receiving at least one network-based communication regarding strain-related characterizing selections includes at least one of the following: at least one classification label, at least one desired effect, at least one side effect, and at least one medical benefit.

10. The method of claim 8, wherein the generating at least one ad hoc matching calculation weighting system based at least in part upon the receiving at least one network-based communication regarding strain-related characterizing selections includes
compiling a reference_profile data vector from the receiving at least one network-based communication regarding strain-related characterizing selections; and
performing a mapping comparison between the reference_profile data vector and each of a plurality of candidate strain_profile data vectors to determine vector differences for each of the plurality of candidate strain_profile data vectors.

11. The method of claim 8, further comprising:
receiving at least one network-based communication regarding network protocol information related to an electronic device geographical location;
determining the electronic device geographical location based on the network protocol information;
comparing the electronic device geographical location to a geographical location associated with at least one candidate strain according to geographical location data stored in the at least one database to determine acceptability of the geographical location associated with the at least one candidate strain; and
outputting at least one network-based communication regarding at least one strain recommendation based at least in part on the acceptability of the geographical location associated with the at least one candidate strain.

12. The method of claim 8, wherein the receiving at least one network-based communication regarding strain-related characterizing selections includes:
receiving user_markers associated at least in part with at least one magnitude of strain-related characterizing selections.

13. The method of claim 8, further comprising:
generating the at least one ad hoc matching calculation weighting system based at least in part upon at least one rule that governs at least one relationship between cannabinoid_profiles and at least one characteristic, the at least one characteristic selected from one or more of at least one Desired Effect, at least one Side Effect, and at least one Medical Benefit.

14. The method of claim 8, further comprising:
modifying characteristic data of at least one strain profile based at least in part upon the receiving at least one network-based communication regarding strain-related characterizing selections.

15. At least one non-transitory computer-readable storage media, having computer executable instructions embodied thereon and configured to run on the at least one processor to cause the at least one processor to perform:
receiving at least one network-based communication regarding strain-related characterizing selections;
generating at least one ad hoc matching calculation weighting system based at least in part upon the receiving at least one network-based communication regarding strain-related characterizing selections;
using the at least one ad hoc matching calculation weighting system with strain candidate data stored in the at least one database to at least in part determine at least one strain candidate score for at least one strain candidate; and
outputting at least one network-based communication regarding at least one strain recommendation based at least in part on the at least one strain candidate score.

16. The at least one non-transitory computer-readable storage media of claim 15, wherein the
computer executable instructions embodied thereon and configured to run on the at least one processor to cause the at least one processor to perform the receiving at least one network-based communication regarding strain-related characterizing selections including at least one of the following: at least one classification label, at least one desired effect, at least one side effect, and at least one medical benefit.

17. The at least one non-transitory computer-readable storage media of claim 15, wherein the
computer executable instructions embodied thereon and configured to run on the at least one processor to cause the at least one processor to perform including
the generating at least one ad hoc matching calculation weighting system based at least in part upon the receiving at least one network-based communication regarding strain-related characterizing selections further including
compiling a reference_profile data vector from the receiving at least one network-based communication regarding strain-related characterizing selections; and
performing a mapping comparison between the reference_profile data vector and each of a plurality of candidate strain_profile data vectors to determine vector differences for each of the plurality of candidate strain_profile data vectors.

18. The at least one non-transitory computer-readable storage media of claim 15, wherein the
computer executable instructions embodied thereon and configured to run on the at least one processor to cause the at least one processor to perform including
receiving at least one network-based communication regarding network protocol information related to an electronic device geographical location;
determining the electronic device geographical location based on the network protocol information;
comparing the electronic device geographical location to a geographical location associated with at least one candidate strain according to geographical location data stored in the at least one database to determine acceptability of the geographical location associated with the at least one candidate strain; and
outputting at least one network-based communication regarding at least one strain recommendation based at least in part on the acceptability of the geographical location associated with the at least one candidate strain.

19. The at least one non-transitory computer-readable storage media of claim 15, wherein the
computer executable instructions embodied thereon and configured to run on the at least one processor to cause the at least one processor to perform the receiving at least one network-based communication regarding strain-related characterizing selections includes
receiving user_markers associated at least in part with at least one magnitude of strain-related characterizing selections.

20. The at least one non-transitory computer-readable storage media of claim 15, wherein the
computer executable instructions embodied thereon and configured to run on the at least one processor to cause the at least one processor to perform including
generating the at least one ad hoc matching calculation weighting system based at least in part upon at least one rule that governs at least one relationship between cannabinoid_profiles and at least one characteristic, the at least one characteristic selected from one or more of at least one Desired Effect, at least one Side Effect, and at least one Medical Benefit.
